(19) United States
(12) Patent Application Publication
(10) Pub. No.: US 2007/0185090 A1
(43) Pub. Date: Aug. 9, 2007

(54) Muscarinic acetylchoine receptor antagonists

(76) Inventors: Jakob Busch-Petersen, King of Prussia, PA (US); Anthony W.J. Cooper, Hertfordshire (GB); Roderick S. Davis, King of Prussia, PA (US); Dramane I. Laine, King of Prussia, PA (US); Michael R. Palovich, King of Prussia, PA (US); Wei Fu, King of Prussia, PA (US)

Correspondence Address: SMITHKLINE BEECHAM CORPORATION;CORPORATE INTELLECTUAL PROPERTY-US, UW2220, P. O. BOX 1539, KING OF PRUSSIA, PA, 19406-0939, US

(21) Appl. No.: 10/598,887

(22) PCT Filed: Mar. 17, 2004
(86) PCT No.: PCT/US04/08026

§ 371 Date: Sep. 14, 2006

(51) Int. Cl. A61K 31/55; C07D 223/16

(52) U.S. Cl. 514/217.02; 540/595

(57) ABSTRACT

Muscarinic Acetylcholine receptor antagonists and methods of using them are provided.

### FIELD OF THE INVENTION

This invention relates to novel benzazepine compounds, pharmaceutical compositions, processes for their preparation, and use thereof in treating muscarinic acetylcholine receptor mediated diseases.

### BACKGROUND OF THE INVENTION

Acetylcholine released from cholinergic neurons in the peripheral and central nervous systems affects many different biological processes through interaction with two major classes of acetylcholine receptors—the nicotinic and the muscarinic acetylcholine receptors. Muscarinic acetylcholine receptors (mAChRs) belong to the superfamily of G-protein coupled receptors that have seven transmembrane domains. There are five subtypes of mAChRs, termed M₁-M₅, and each is the product of a distinct gene. Each of these five subtypes displays unique pharmacological properties. Muscarinic acetylcholine receptors are widely distributed in vertebrate organs, and these receptors can mediate both inhibitory and excitatory actions. For example, in smooth muscle found in the airways, bladder and gastrointestinal tract, M₃ mAChRs mediate contractile responses (1989. The Muscarinic Receptors. The Humana Press, Inc., Clifton, N.J.).

Muscarinic acetylcholine receptor dysfunction has been noted in a variety of different pathophysiological states. For instance, in asthma and chronic obstructive pulmonary disease (COPD), inflammatory conditions lead to loss of inhibitory M₂ muscarinic acetylcholine autoreceptor function on parasympathetic nerves supplying the pulmonary smooth muscle, causing increased acetylcholine release following vagal nerve stimulation. This mAChR dysfunction results in airway hyperreactivity mediated by increased stimulation of M₃ mAChRs. Similarly, inflammation of the gastrointestinal tract in inflammatory bowel disease (IBD) results in M₃ mAChR-mediated hypermotility (Oprins, J. C. J., H P. Meijer, and J. A. Groot. 2000. Tumor Necrosis Factor-{alpha} Potentiates Ion Secretion Induced by Muscarinic Receptor Activation in the Human Intestinal Epithelial Cell Line HT29cl.19A. Ann NY Acad Sci 915:102-106). Incontinence due to bladder hypercontractility has also been demonstrated to be mediated through increased stimulation of M₃ mAChRs. Thus the identification of subtytpe-selective mAChR antagonists may be useful as therapeutics in these mAChR-mediated diseases.

Despite the large body of evidence supporting the use of anti-muscarinic receptor therapy for treatment of a variety of disease states, relatively few anti-muscarinic compounds are in use in the clinic. Thus, there remains a need for novel compounds that are capable of causing blockade at M₃ mAChRs. Conditions associated with an increase in stimulation of M₃ mAChRs, such as asthma, COPD, IBD and urinary incontinence would benefit by compounds that are inhibitors of mAChR binding.

### SUMMARY OF THE INVENTION

This invention provides for a method of treating a muscarinic acetylcholine receptor (mAChR) mediated disease, wherein acetylcholine binds to an mAChR and which method comprises administering an effective amount of a compound of Formula (I) or a pharmaceutically acceptable salt thereof.

This invention also relates to a method of inhibiting the binding of acetylcholine to its receptors in a mammal in need thereof which comprises administering to aforementioned mammal an effective amount of a compound of Formula (I).

The present invention also provides for the novel compounds of Formula (I), and pharmaceutical compositions comprising a compound of Formula (I), and a pharmaceutical carrier or diluent; 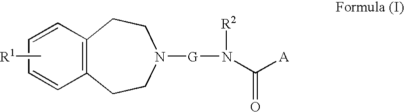
wherein:

R¹ is selected from the group consisting of a substituent selected from: C₁₋₆ alkanoyl, aroyl and aroylC₁₋₆alkyl, all optionally substituted;

R² is selected from the group consisting of a hydrogen atom or a C₁₋₄alkyl group;
G is selected from the group consisting of C₄₋₇alkyl or a group of the formula (a), (b), (c) or (d) 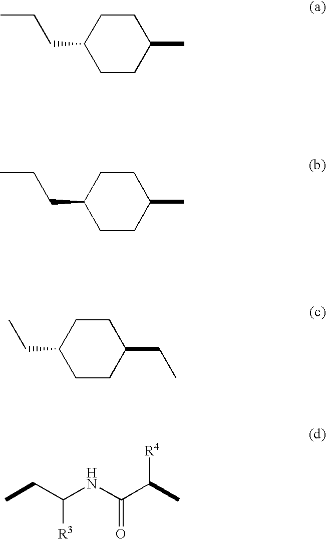
   Wherein,

R³ and R⁴ are, independently, selected from a group consisting of a hydrogen, C₁₋₄alkyl, aryl, or C₁₋₂alkylaryl;
A is selected from a group consisting of an optionally substituted C₁₋₆alkyl or a group of the formula (e), (f), (g) or (h): 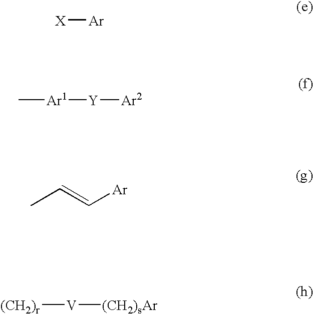
   wherein
X is selected from a group consisting of a bond, NR², O or S;
Ar is selected from a group consisting of an optionally substituted phenyl ring or an optionally substituted 5- or 6-membered aromatic heterocyclic ring; or an optionally substituted bicyclic or heterobicyclic ring system;
Ar¹ and Ar² each independently is selected from a group consisting of an optionally substituted phenyl ring or an optionally substituted 5- or 6-membered aromatic heterocyclic ring; and
Y is selected from a group consisting of a bond, —NHCO—, —CONH—, —CH₂—, or —(CH₂)ₘY¹ (CH₂)ₙ—, wherein Y¹ is selected from a group consisting of O, S, SO₂, or CO and m and n each represent zero or 1 such that the sum of m+n is zero or 1; provided that when A represents a group of formula (a), any substituent present in Ar ortho to the carboxamide moiety is necessarily a hydrogen or a methoxy group;
r and s are, independently, selected from a group consisting of an integer from zero to 3 such that the sum of r and s is equal to an integer from 1 to 6;

V is selected from a group consisting of a bond, O, S, —NHCO—, —CONH—, CHNHCOR³;

and salts thereof.

In the compounds of formula (I) above an alkyl group or moiety may be straight or branched. Alkyl groups which may be employed include methyl, ethyl, n-propyl, n-butyl, n-pentyl, n-hexyl and any branched isomers thereof such as isopropyl, t-butyl, sec-butyl, and the like.

When R¹ is selected from a group consisting of an aroyl, or aroylC₁₋₄alkyl, the aryl moiety may be selected from an optionally substituted phenyl ring or an optionally substituted 5- or 6-membered heterocyclic ring. In the group R¹ an aryl moiety may be optionally substituted by one or more substituents selected from hydrogen, halogen, amino, cyano, C₁₋₄alkyl, C₁₋₄alkylamino, C₁₋₄dialkylamino, C₁₋₄alkylamido, C₁₋₄alkanoyl, or R⁵R⁶NCO where each of R⁵ and R⁶ are, independently, selected from a group consisting of a hydrogen atom or C₁₋₄alkyl group.

An optionally substituted 5- or 6-membered heterocyclic aromatic ring, as defined for any of the groups Ar, Ar¹ or Ar² may contain from 1 to 4 heteroatoms selected from O, N or S. When the ring contains 2-4 heteroatoms, one is preferably selected from O, N and S and the remaining heteroatoms are preferably N. Examples of 5 and 6-membered heterocyclic groups include furyl, thienyl, pyrrolyl, oxazolyl, thiazolyl, imidazolyl, oxadiazolyl, thiadiazolyl, pyridyl, triazolyl, triazinyl, pyridazyl, pyrimidinyl, pyrazolyl, isothiazolyl, and isoxazolyl.

Examples of bicyclic, for example bicyclic aromatic or heteroaromatic, ring systems for Ar include naphthyl, indazolyl, indolyl, benzofuranyl, benzothienyl, benzothiazolyl, benzimidazolyl, benzoxazolyl, benzisoxazolyl, benzisothiazolyl, quinolinyl, quinoxolinyl, quinazolinyl, cinnolinyl, isoquinolinyl, pyrazolo[1,5-a]pyrimidyl, pyrrolo[3,2-b]pyridyl, pyrrolo[3,2-c]pyridyl, thieno[3,2-b]thiophenyl, 1,2-dihydro-2-oxo-quinolinyl, 3,4-dihydro-3-oxo-2H-benzoxazinyl, 1,2-dihydro-2-oxo-3H-indolyl.

The rings Ar, Ar¹, or Ar² may each independently be optionally substituted by one or more substituents selected from: a hydrogen or halogen atom, or a hydroxy, oxo, cyano, nitro, trifluoromethyl, C₁₋₄alkyl, C₁₋₄alkoxy, C₁₋₄alkylenedioxy, C₁₋₄alkanoyl, C₁₋₄alkylsulfonyl, C₁₋₄alkylsulfinyl, C₁₋₄alkylthio, R⁷SO₂N(R⁸)—, R⁷R⁸NSO₂—, R⁷R⁸N—, R⁷O₂C—, R⁷R⁸NC(O)—, or R⁷CON(R⁸)— group wherein each of R⁷ and R⁸ independently, selected from a group consisting of a hydrogen atom or a C₁₋₄ alkyl group, or R⁷R⁸ together form a C₃₋₆ alkylene chain.

Alternatively, Ar and Ar² may be optionally substituted by one or more 5- or 6-membered heterocyclic rings, as defined above, optionally substituted by a C₁₋₂ alkyl or R⁷R⁸N— group; wherein R⁷ and R⁸ are as defined above.

In the rings Ar and Ar² substituents positioned ortho to one another may be linked to form a 5- or 6-membered ring.

It will be appreciated that for use in medicine the salts of formula (I) should be physiologically acceptable. Suitable physiologically acceptable salts will be apparent to those skilled in the art and include for example acid addition salts formed with inorganic acids eg. hydrochloric, hydrobromic, sulfuric, nitric or phosphoric acid; and organic acids eg. succinic, maleic, acetic, fumaric, citric, tartaric, benzoic, p-toluenesulfonic, methanesulfonic or naphthalenesulfonic acid. Other non-physiologically acceptable salts eg. oxalates, may be used, for example in the isolation of compounds of formula (I) and are included within the scope of this invention. Also included within the scope of the invention are solvates and hydrates of compounds of formula (I).

Certain of the compounds of formula (I) may form acid addition salts with one or more equivalents of the acid. The present invention includes within its scope all possible stoichiometric and non-stoichiometric forms.

The compounds of formula (I) can exist in the form of cis- and trans-isomers with respect to the configuration at the cyclohexyl ring. When A represents a group (c) the compounds may also exist as geometric isomers around the double bond. The present invention includes within its scope all such isomers, including mixtures. Preferably the compounds of the invention are in the trans configuration with respect to the cyclohexyl ring. For compounds of formula (I) where A represents a group (c), trans geometry of the double bond is preferred.

In compounds of formula (I), it is preferred that R¹ represents a substituent selected from: a halogen atom, methyl, cyano, acetyl, trifluoromethyl, pentafluoroethyl, methylsulphonyl, methylsulphonyloxy or trifluoromethoxy group. Alternatively, it is preferred that R¹ represents a group Ar³Z, where Z is a bond and Ar³ is a 5- or 6-membered ring heterocycle, optionally substituted by a methyl group, containing at least one N and one O atom. R² is preferably a hydrogen atom.

When the group A is a group of formula (a), preferred examples of Ar include optionally substituted phenyl, indolyl, pyrazolo[1,5-a]pyrimidyl, cinnolinyl, quinolinyl, benzo[b]furanyl or pyrrolopyridyl.

When the group A is a group of formula (b), preferred examples of Ar¹ include optionally substituted phenyl, Y is preferably a bond, and preferred examples of Ar² include optionally substituted phenyl, pyridyl, pyrimidinyl, isoxazolyl, oxazolyl or oxadiazolyl.

When the group A is a group of formula (c), preferred examples of Ar include optionally substituted phenyl.

It is also preferred that the rings Ar, Ar¹, or Ar² are each independently optionally substituted by one or more substituents selected from: a hydrogen or halogen atom, cyano, methoxy, trifluoromethyl, methylenedioxy, acetyl, acetylamino, methylsulfonyl, methylsulfonyloxy, methylaminosulfonyl, methylsulfonylamino, or methylaminocarbonyl group.

Certain of the substituted heteroaromatic ring systems included in compounds of formula (I) may exist in one or more tautomeric forms. The present invention includes within its scope all such tautomeric forms, including mixtures. The following terms, as used herein, refer to:
“halo”—all halogens, that is chloro, fluoro, bromo and iodo.
“C₁₋₁₀alkyl” or “alkyl”—both straight and branched chain moieties of 1 to 10 carbon atoms, unless the chain length is otherwise limited, including, but not limited to, methyl, ethyl, n-propyl, iso-propyl, n-butyl, sec-butyl, iso-butyl, tert-butyl, n-pentyl and the like.
“cycloalkyl” is used herein to mean cyclic moiety, preferably of 3 to 8 carbons, including but not limited to cyclopropyl, cyclopentyl, cyclohexyl, and the like.
“alkenyl” is used herein at all occurrences to mean straight or branched chain moiety of 2-10 carbon atoms, unless the chain length is limited thereto, including, but not limited to ethenyl, 1-propenyl, 2-propenyl, 2-methyl-1-propenyl, 1-butenyl, 2-butenyl and the like.
“aryl”—phenyl and naphthyl;
“heteroaryl” (on its own or in any combination, such as “heteroaryloxy”, or “heteroaryl alkyl”)—a 5-10 membered aromatic ring system in which one or more rings contain one or more heteroatoms selected from the group consisting of N, O or S, such as, but not limited, to pyrrole, pyrazole, furan, thiophene, quinoline, isoquinoline, quinazolinyl, pyridine, pyrimidine, oxazole, tetrazole, thiazole, thiadiazole, triazole, imidazole, or benzimidazole.
“heterocyclic” (on its own or in any combination, such as “heterocyclicalkyl”)—a saturated or partially unsaturated 4-10 membered ring system in which one or more rings contain one or more heteroatoms selected from the group consisting of N, O, or S; such as, but not limited to, pyrrolidine, piperidine, piperazine, morpholine, tetrahydropyran, thiomorpholine, or imidazolidine. Furthermore, sulfur may be optionally oxidized to the sulfone or the sulfoxide.
“arylalkyl” or “heteroarylalkyl” or “heterocyclicalkyl” is used herein to mean C₁₋₁₀alkyl, as defined above, attached to an aryl, heteroaryl or heterocyclic moiety, as also defined herein, unless otherwise indicated.

Particular compounds according to the invention include those specifically exemplified and named hereinafter, Most preferred:
2,8-dimethyl-N-(trans-4-{2-[7-(2-methylpropanoyl)-1,2,4,5-tetrahydro-3H-3-benzazepin-3-yl]ethyl}cyclohexyl)-5-quinolinecarboxamide;
2,8-Dimethyl-quinoline-5-carboxylic acid{4-[2-(7-butyryl-1,2,4,5-tetrahydro-3H-3-benzazepin-3-yl)-ethyl]-cyclohexyl}-amide;
8-Methoxy-2-methyl-quinoline-5-carboxylic acid{4-[2-(7-butyryl-1,2,4,5-tetrahydro-3H-3-benzazepin3-yl)-ethyl]-cyclohexyl}-amide;
8-Chloro-2-methyl-quinoline-5-carboxylic acid{4-[2-(7-butyryl-1,2,4,5-tetrahydro-3H-3-benzazepin3-yl)-ethyl]-cyclohexyl}-amide;
N-(trans-4-{2-[7-(2-methylpropanoyl)-1,2,4,5-tetrahydro-3H-3-benzazepin-3-yl]ethyl}cyclohexyl)-5-quinoxalinecarboxamide;
N-(trans-4-{2-[7-(2-methylpropanoyl)-1,2,4,5-tetrahydro-3H-3-benzazepin-3-yl]ethyl}cyclohexyl)-2,2-diphenylacetamide;
2-Methyl-quinoline-5-carboxylic acid{4-[2-(7-butyryl-1,2,4,5-tetrahydro-3H-3-benzazepin3-yl)-ethyl]-cyclohexyl}-amide;
N-(trans-4-{2-[7-(2-methylpropanoyl)-1,2,4,5-tetrahydro-3H-3-benzazepin-3-yl]ethyl}cyclohexyl)-1H-indole-2-carboxamide;
1,1-dimethylethyl 2-{[(trans-4-{2-[7-(2-methylpropanoyl)-1,2,4,5-tetrahydro-3H-3-benzazepin-3-yl]ethyl}cyclohexyl)amino]carbonyl}benzoate;
8-chloro-2-methyl-N-(trans-4-{2-[7-(2-methylpropanoyl)-1,2,4,5-tetrahydro-3H-3-benzazepin-3-yl]ethyl}cyclohexyl)-5-quinolinecarboxamide;
2-methyl-8-(methyloxy)-N-(trans-4-{2-[7-(2-methylpropanoyl)-1,2,4,5-tetrahydro-3H-3-benzazepin-3-yl]ethyl}cyclohexyl)-5-quinolinecarboxamide;
8-chloro-N-(trans-4-{2-[7-(2-methylpropanoyl)-1,2,4,5-tetrahydro-3H-3-benzazepin-3-yl]ethyl}cyclohexyl)-5-quinolinecarboxamide;
   Preferred:
1,1-dimethylethyl((1S)-2-[(trans-4-{2-[7-(2-methylpropanoyl)-1,2,4,5-tetrahydro-3H-3-benzazepin-3-yl]ethyl}cyclohexyl)amino]-2-oxo-1-{[(phenylmethyl)oxy]methyl}ethyl)carbamate formate;
N-(trans-4-{2-[7-(2-methylpropanoyl)-1,2,4,5-tetrahydro-3H-3-benzazepin-3-yl]ethyl}cyclohexyl)-N′-(phenylmethyl)butanediamide;
N-{5-[(trans-4-{2-[7-(2-methylpropanoyl)-1,2,4,5-tetrahydro-3H-3-benzazepin-3-yl]ethyl}cyclohexyl)amino]-5-oxopentyl}benzamide;
N-(trans-4-{2-[7-(2-methylpropanoyl)-1,2,4,5-tetrahydro-3H-3-benzazepin-3-yl]ethyl}cyclohexyl)-2-[(methylsulfonyl)amino]benzamide;
N¹-(trans-4-{2-[7-(2-methylpropanoyl)-1,2,4,5-tetrahydro-3H-3-benzazepin-3-yl]ethyl}cyclohexyl)-O-(phenylmethyl)-L-serinamide;
N-(trans-4-{2-[7-(2-methylpropanoyl)-1,2,4,5-tetrahydro-3H-3-benzazepin-3-yl]ethyl}cyclohexyl)-1-isoquinolinecarboxamide formate;
N-(trans-4-{2-[7-(2-methylpropanoyl)-1,2,4,5-tetrahydro-3H-3-benzazepin-3-yl]ethyl}cyclohexyl)-1,6-naphthyridine-2-carboxamide;
1,1-dimethylethyl({3-[(trans-4-{2-[7-(2-methylpropanoyl)-1,2,4,5-tetrahydro-3H-3-benzazepin-3-yl]ethyl}cyclohexyl)amino]-3-oxopropyl}thio)acetate;
2-(dimethylamino)-N-(trans-4-{2-[7-(2-methylpropanoyl)-1,2,4,5-tetrahydro-3H-3-benzazepin-3-yl]ethyl}cyclohexyl)benzamide;
N-(trans-4-{2-[7-(2-methylpropanoyl)-1,2,4,5-tetrahydro-3H-3-benzazepin-3-yl]ethyl}cyclohexyl)-3,3-diphenylpropanamide;
N-(trans-4-{2-[7-(2-methylpropanoyl)-1,2,4,5-tetrahydro-3H-3-benzazepin-3-yl]ethyl}cyclohexyl)-2-(2-naphthalenyl)acetamide formate;
(2E)-3-(1H-imidazol-4-yl)-N-(trans-4-{2-[7-(2-methylpropanoyl)-1,2,4,5-tetrahydro-3H-3-benzazepin-3-yl]ethyl}cyclohexyl)-2-propenamide;
N-(trans-4-{2-[7-(2-methylpropanoyl)-1,2,4,5-tetrahydro-3H-3-benzazepin-3-yl]ethyl}cyclohexyl)-6-(1H-pyrrol-1-yl)-3-pyridinecarboxamide;
4-amino-N-(trans-4-{2-[7-(2-methylpropanoyl)-1,2,4,5-tetrahydro-3H-3-benzazepin-3-yl]ethyl}cyclohexyl)butanamide;
N-(trans-4-{2-[7-(2-methylpropanoyl)-1,2,4,5-tetrahydro-3H-3-benzazepin-3-yl]ethyl}cyclohexyl)-4-pyridinecarboxamide formate;
1,1-dimethylethyl {4-[(trans-4-{2-[7-(2-methylpropanoyl)-1,2,4,5-tetrahydro-3H-3-benzazepin-3-yl]ethyl}cyclohexyl)amino]-4-oxobutyl}carbamate;
3-[3,4-bis(methyloxy)phenyl]-N-(trans-4-{2-[7-(2-methylpropanoyl)-1,2,4,5-tetrahydro-3H-3-benzazepin-3-yl]ethyl}cyclohexyl)propanamide;
N-{2-[(trans-4-{2-[7-(2-methylpropanoyl)-1,2,4,5-tetrahydro-3H-3-benzazepin-3-yl]ethyl}cyclohexyl)amino]-2-oxoethyl}-3-pyridinecarboxamide formate;
N-(trans-4-{2-[7-(2-methylpropanoyl)-1,2,4,5-tetrahydro-3H-3-benzazepin-3-yl]ethyl}cyclohexyl)-1-phenylcyclopentanecarboxamide;
2-[4-(dimethylamino)phenyl]-N-(trans-4-{2-[7-(2-methylpropanoyl)-1,2,4,5-tetrahydro-3H-3-benzazepin-3-yl]ethyl}cyclohexyl)acetamide;
N-(trans-4-{2-[7-(2-methylpropanoyl)-1,2,4,5-tetrahydro-3H-3-benzazepin-3-yl]ethyl}cyclohexyl)-1,8-naphthyridine-2-carboxamide;
N-(trans-4-{2-[7-(2-methylpropanoyl)-1,2,4,5-tetrahydro-3H-3-benzazepin-3-yl]ethyl}cyclohexyl)-2-(4-pyridinyl)acetamide;
N-(trans-4-{2-[7-(2-methylpropanoyl)-1,2,4,5-tetrahydro-3H-3-benzazepin-3-yl]ethyl}cyclohexyl)-4-oxo-4-phenyl-2-butenamide;
2-(5-hydroxy-1H-benzimidazol-1-yl)-N-(trans-4-{2-[7-(2-methylpropanoyl)-1,2,4,5-tetrahydro-3H-3-benzazepin-3-yl]ethyl}cyclohexyl)acetamide;
N-(trans-4-{2-[7-(2-methylpropanoyl)-1,2,4,5-tetrahydro-3H-3-benzazepin-3-yl]ethyl}cyclohexyl)-2-pyrazinecarboxamide;
1-methyl-N-(trans-4-{2-[7-(2-methylpropanoyl)-1,2,4,5-tetrahydro-3H-3-benzazepin-3-yl]ethyl}cyclohexyl)-1H-indole-2-carboxamide;
N-(trans-4-{2-[7-(2-methylpropanoyl)-1,2,4,5-tetrahydro-3H-3-benzazepin-3-yl]ethyl}cyclohexyl)-2-(3-pyridinyl)-1,3-thiazole-4-carboxamide;
1,1-dimethylethyl {(1R)-2-[(trans-4-{2-[7-(2-methylpropanoyl)-1,2,4,5-tetrahydro-3H-3-benzazepin-3-yl]ethyl}cyclohexyl)amino]-2-oxo-1-phenylethyl}carbamate;
N-(trans-4-{2-[7-(2-methylpropanoyl)-1,2,4,5-tetrahydro-3H-3-benzazepin-3-yl]ethyl}cyclohexyl)-3-pyridinecarboxamide 1-oxide formate;
3-(dimethylamino)-N-(trans-4-{2-[7-(2-methylpropanoyl)-1,2,4,5-tetrahydro-3H-3-benzazepin-3-yl]ethyl}cyclohexyl)benzamide;
N-(trans-4-{2-[7-(2-methylpropanoyl)-1,2,4,5-tetrahydro-3H-3-benzazepin-3-yl]ethyl}cyclohexyl)-1H-indole-2-carboxamide;
3-(1H-indol-3-yl)-N-(trans-4-{2-[7-(2-methylpropanoyl)-1,2,4,5-tetrahydro-3H-3-benzazepin-3-yl]ethyl}cyclohexyl)propanamide;
N-(trans-4-{2-[7-(2-methylpropanoyl)-1,2,4,5-tetrahydro-3H-3-benzazepin-3-yl]ethyl}cyclohexyl)-2-quinolinecarboxamide;
N-(trans-4-{2-[7-(2-methylpropanoyl)-1,2,4,5-tetrahydro-3H-3-benzazepin-3-yl]ethyl}cyclohexyl)-2-(1H-pyrrol-1-yl)benzamide;
1,1-dimethylethyl [(1R)-2-[(trans-4-{2-[7-(2-methylpropanoyl)-1,2,4,5-tetrahydro-3H-3-benzazepin-3-yl]ethyl}cyclohexyl)amino]-2-oxo-1-(3-pyridinylmethyl)ethyl]carbamate formate;
N-(trans-4-{2-[7-(2-methylpropanoyl)-1,2,4,5-tetrahydro-3H-3-benzazepin-3-yl]ethyl}cyclohexyl)-1H-indole-3-carboxamide formate;
5-methyl-N-(trans-4-{2-[7-(2-methylpropanoyl)-1,2,4,5-tetrahydro-3H-3-benzazepin-3-yl]ethyl}cyclohexyl)-2-phenyl-2H-1,2,3-triazole-4-carboxamide;
N³— [(4-methylphenyl)sulfonyl]-N¹-(trans-4-{2-[7-(2-methylpropanoyl)-1,2,4,5-tetrahydro-3H-3-benzazepin-3-yl]ethyl}cyclohexyl)-beta-alaninamide formate;
(2R)-2-amino-N-(trans-4-{2-[7-(2-methylpropanoyl)-1,2,4,5-tetrahydro-3H-3-benzazepin-3-yl]ethyl}cyclohexyl)-2-phenylethanamide;
N-(trans-4-{2-[7-(2-methylpropanoyl)-1,2,4,5-tetrahydro-3H-3-benzazepin-3-yl]ethyl}cyclohexyl)-2-(2-pyrimidinylthio)acetamide formate;
N-(trans-4-{2-[7-(2-methylpropanoyl)-1,2,4,5-tetrahydro-3H-3-benzazepin-3-yl]ethyl}cyclohexyl)-4-(1H-pyrrol-1-yl)benzamide;
N¹-(trans-4-{2-[7-(2-methylpropanoyl)-1,2,4,5-tetrahydro-3H-3-benzazepin-3-yl]ethyl}cyclohexyl)-3-(3-pyridinyl)-D-alaninamide;
(3E)-N-(trans-4-{2-[7-(2-methylpropanoyl)-1,2,4,5-tetrahydro-3H-3-benzazepin-3-yl]ethyl}cyclohexyl)-4-phenyl-3-butenamide;
2-(1H-indol-3-yl)-N-(trans-4-{2-[7-(2-methylpropanoyl)-1,2,4,5-tetrahydro-3H-3-benzazepin-3-yl]ethyl}cyclohexyl)acetamide;
N-(trans-4-{2-[7-(2-methylpropanoyl)-1,2,4,5-tetrahydro-3H-3-benzazepin-3-yl]ethyl}cyclohexyl)-2-[(phenylmethyl)thio]acetamide;
4-[4-(methyloxy)phenyl]-N-(trans-4-{2-[7-(2-methylpropanoyl)-1,2,4,5-tetrahydro-3H-3-benzazepin-3-yl]ethyl}cyclohexyl)butanamide;
2-Methyl-quinoline-5-carboxylic acid{4-[7-(2-methyl-propanoyl)-1,2,4,5-tetrahydro-3H-3-benzazepin-3-yl]-butyl}-amide;
2-Methyl-quinoline-5-carboxylic acid{8-[7-(2-methyl-propanoyl)-1,2,4,5-tetrahydro-3H-3-benzazepin-3-yl]-octyl}-amide;
2-Methyl-quinoline-5-carboxylic acid [({4-[7-(2-methyl-propanoyl)-1,2,4,5-tetrahydro-3H-3-benzazepin-3-ylmethyl]-cyclohexylmethyl}-carbamoyl)-methyl]-amide;
(R)-1-[1-(2-Methyl-quinolin-5-yl)-methanoyl)-pyrrolidine-2-carboxylic acid{4-[7-(2-methyl-propanoyl)-1,2,4,5-tetrahydro-3H-3-benzazepin-3-ylmethyl]-cyclohexylmethyl}-amide2-Methyl-quinoline-5-carboxylic acid;
[2-({4-[7-(2-methyl-propanoyl)-1,2,4,5-tetrahydro-3H-3-benzazepin-3-ylmethyl]-cyclohexylmethyl}-carbamoyl)-ethyl]-amide;
2,8-Dimethyl-quinoline-5-carboxylic acid{4-[2-(7-butyryl-1,2,4,5-tetrahydro-3H-3-benzazepin-3-yl)-ethyl]-cyclohexyl}-amide;
8-Methoxy-2-methyl-quinoline-5-carboxylic acid{4-[2-(7-butyryl-1,2,4,5-tetrahydro-3H-3-benzazepin3-yl)-ethyl]-cyclohexyl}-amide;
8-Chloro-2-methyl-quinoline-5-carboxylic acid{4-[2-(7-butyryl-1,2,4,5-tetrahydro-3H-3-benzazepin3-yl)-ethyl]-cyclohexyl}-amide;
2-Methyl-quinoline-5-carboxylic acid{4-[2-(7-butyryl-1,2,4,5-tetrahydro-3H-3-benzazepin3-yl)-ethyl]-cyclohexyl}-amide;
N-{4-[2-(7-Acetyl-1,2,4,5-tetrahydro-3H-3-benzazepin-3-yl)-ethyl]-cyclohexyl}-4-fluoro-benzamide;
N-{4-[2-(7-Acetyl-1,2,4,5-tetrahydro-3H-3-benzazepin-3-yl)-ethyl]-cyclohexyl}-3-(5-methyl-[1,2,4]oxadiazol-3-yl)-benzamide;
2-Methyl-quinoline-5-carboxylic acid{4-[2-(7-(2-methyl-propanoyl)-1,2,4,5-tetrahydro-3H-3-benzazepin3-yl)-ethyl]-cyclohexyl}-amide;
1H-pyrrolo[2,3-b]pyridine-3-carboxylic acid-{4-[2-(7-Acetyl-1,2,4,5-tetrahydro-3H-3-benzazepin-3-yl)-ethyl]-cyclohexyl}-amide;
(E)-N-{4-[2-(7-Acetyl-1,2,4,5-tetrahydro-3H-3-benzazepin-3-yl)-ethyl]-cyclohexyl}-3-(4-fluorophenyl)-acrylamide;
(E)-N-{4-[2-(7-Acetyl-1,2,4,5-tetrahydro-3H-3-benzazepin-3-yl)-ethyl]-cyclohexyl}-3-(3-methoxyphenyl)-acrylamide;
(E)-N-{4-[2-(7-Acetyl-1,2,4,5-tetrahydro-3H-3-benzazepin-3-yl)-ethyl]-cyclohexyl}-3-(2-cyanophenyl)-acrylamide;
(E)-N-{4-[2-(7-Acetyl-1,2,4,5-tetrahydro-3H-3-benzazepin-3-yl)-ethyl]-cyclohexyl}-3-(2-acetylphenyl)-acrylamide;
(E)-N-{4-[2-(7-Acetyl-1,2,4,5-tetrahydro-3H-3-benzazepin-3-yl)-ethyl]-cyclohexyl}-3-(3-thiophenyl)-acrylamide;
(E)-N-{4-[2-(7-Acetyl-1,2,4,5-tetrahydro-3H-3-benzazepin-3-yl)-ethyl]-cyclohexyl}-3-(8-(1,2-dihydro-2-oxo)-quinolinyl)-acrylamide;
(E)-N-{4-[2-(7-Acetyl-1,2,4,5-tetrahydro-3H-3-benzazepin-3-yl)-ethyl]-cyclohexyl}-3-(3-benzothiophenyl)-acrylamide;
(E)-N-{4-[2-(7-Acetyl-1,2,4,5-tetrahydro-3H-3-benzazepin-3-yl)-ethyl]-cyclohexyl}-3-(3-benzothiophenyl)-acrylamide;
(E)-3-(4-Acetylamino-phenyl)-N-{4-[2-(7-acetyl-1,2,4,5-tetrahydro-3H-3-benzazepin-3-yl)-ethyl]-cyclohexyl}-acrylamide;
N-{4-[2-(7-Acetyl-1,2,4,5-tetrahydro-3H-3-benzazepin-3-yl)-ethyl]-cyclohexyl}-2-(2-aminobenzothiazol-6-yl)-acetamide;
4-Oxo-1,4-dihydro-quinoline-8-carboxylic acid-{4-[2-(7-Acetyl-1,2,4,5-tetrahydro-3H-3-benzazepin-3-yl)-ethyl]-cyclohexyl}-amide;
N-{4-[2-(7-Acetyl-1,2,4,5-tetrahydro-3H-3-benzazepin-3-yl)-ethyl]-cyclohexyl}-2-(benzo[b]thiophen-2-yl)-acetamide and pharmaceutically acceptable salts thereof.

### Methods of Preparation

The compounds of Formula (I) may be obtained by utilizing synthetic procedures, some of which are illustrated in the Schemes below. The synthesis provided for these Schemes is applicable for producing compounds of formula (I) with a variety of different R1, R2, R3, R′ and R″. While the Schemes are shown with compounds only of Formula (I), this is merely for illustration purpose only.

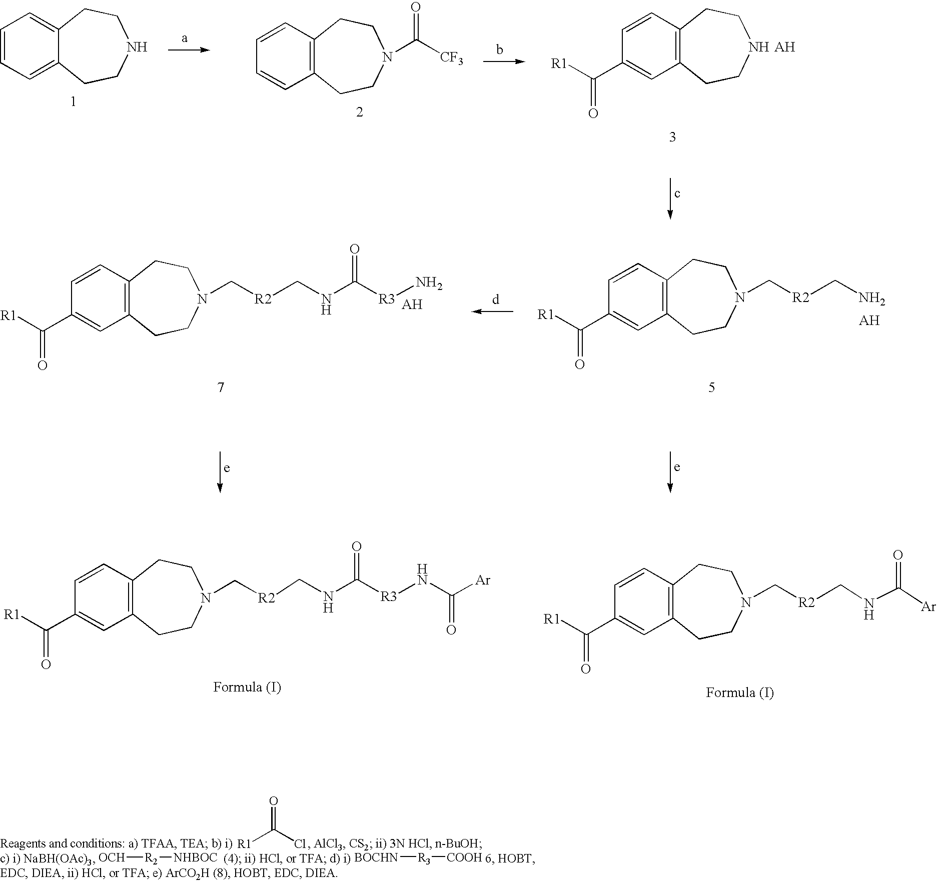

The desired compounds of formula (I) can be prepared as outlined in Scheme 1. The 3H-3-benzazepine 1 was protected to yield trifluoroamide 2 using standard methods well know to those skilled in the art such as treatment with trifluoroacetic anhydride. The alkanoyl-3H-3-benzazepine 3 can be prepared from the corresponding 2 via Friedel-Crafts acylation followed by removal of the trifluoroacetyl group under acidic condition. The amine 5 can be prepared using the well known reductive amination reaction by treating the amines 3 with aldehydes 4 in the presence of a suitable reducing agent such as sodium triacetoxyborohydride. followed by removal of the protecting group using standard conditions well known in the art such as treatment with hydrochloric acid or trifluoroacetic acid. Compounds of Formula (I) can then be obtained by coupling of the amine 5 with a suitable carboxylic acid 8 in the presence of amide coupling reagents such as 1-hydroxybenzotriazole hydrate (HOBT) and 1-(3-dimethylaminopropyl)-3-ethylcarbodiimide (EDC), or 5 be coupled with a suitable protected amino acid 6 gives the intermediate carbamate. This is subsequently deprotected under standard conditions such as treatment with hydrochloric acid or trifluoroacetic acid to yield the amine 7. Finally, coupling of this amine with a suitable carboxylic acid 8 yields the target compound of formula (I). Alternatively, compounds 3 can be prepared according to Hadley et al. (WO 00/21951).

If the required N-protected alkyl aldehyde 4 is not commercially available, it can be prepared from a commercially available acid 9 as illustrated in Scheme 2. Thus, the acid 9 can be reduced to the corresponding alcohol 10 with boron trifluoride-THF complex. Oxidizing this alcohol 10 with pyridinium chlorochromate (PCC) produces the desired aldehyde 4. In the case of R2=1,4-trans-cyclohexyl, the compound 4 can be prepared according to Stemp et al. (J. Med. Chem. 2000, 43, 1878-85).

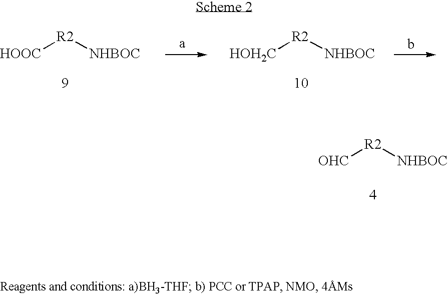

If the required acid 8 is of the quinoline-5-carboxylic acid-type, it can be prepared as outlined in Scheme 3. The 3-amino-benzolic acid 11 can be converted to quinoline-5-carboxylic acid 8 by condensing with a suitable propenal 12. Otherwise, non-commercially available acids 8 can be prepared as described by Hadley et al.

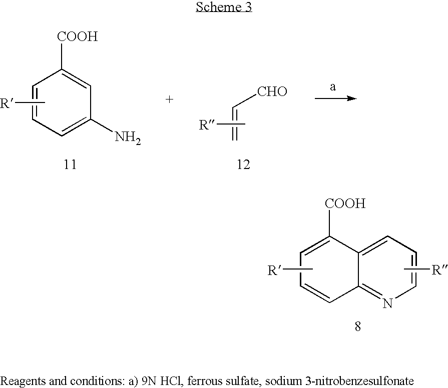

### SYNTHETIC EXAMPLES

### Example 1

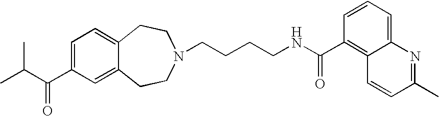

### trans-2-Methyl-quinoline-5-carboxylic acid{4-[7-(2-methyl-propanoyl)-1,2,4,5-tetrahydro-3H-3-benzazepin-3-yl]-butyl}-amide

### 1a) 2,2,2-Trifluoro-1-(1,2,4,5-tetrahydro-3H-3-benzazepin-3-yl)-ethanone

2,3,4,5-tetrahydro-1H-benzo[d]azepine (33.5 g, 228 mmol) was dissolved in 93 mL dichloromethane. Triethylamine (48 mL, 342 mmol) was added and the mixture was cooled on an icebath. TFAA (33.5 mL, 239 mmol) was added dropwise from an addition funnel over 30 min. Stirring was continued overnight while the reaction mixture was allowed to warm to room temperature. The reaction mixture was washed with water, saturated aqueous NaHCO₃ and brine and the combined aqueous layers were extracted with 40 mL dichloromethane. The combined organic layers were dried over magnesium sulfate, filtered and concentrated in vacuo yielding 52.1 g of a pale yellow solid. LCMS: m/z 244 (M+H).

### 1b) 2-Methyl-1-[3-(2,2,2-trifluoro-ethanoyl)-2,3,4,5-tetrahydro-1H-3H-3-benzazepin-7-yl]-propan-1-one

2,2,2-Trifluoro-1-(1,2,4,5-tetrahydro-3H-3-benzazepin-3-yl)-ethanone (4.3 g, 17.69 mmol) was dissolved in 20 mL carbondisulfide. Aluminum chloride (15.1 g, 113 mmol) was added, followed by isobutyryl chloride (5.56 mL, 53.07 mmol) dropwise. The mixture was heated to 45° C. for 1.5 h and then at RT for 30 min. The solvent was evaporated. The residue was dissolved in dichloromethane and cooled in an ice-bath. The extra aluminum chloride was quenched with 6N HCl. The mixture was washed with water (2×). The aquous layer was extracted with CH₂Cl₂ (2×) and the combined organic layers were washed with brine and dried over magnesium sulfate. The solvent was removed in vacuo. The resulting crude was purified via column chromatography on silica gel using ethyl acetate/hexane (20/80, v/v) as mobile phase to give 4.74 g of the title compound (85%). LCMS m/z 314 (M+H).

### 1c) 2-Methyl-1-(2,3,4,5-tetrahydro-1H-3H-3-benzazepin-7-yl)-propan-1-one

2-Methyl-1-[3-(2,2,2-trifluoro-ethanoyl)-2,3,4,5-tetrahydro-1H-3H-3-benzazepin-7-yl]-propan-1-one (4.74 g, 15.14 mmol) was dissolved in 84 mL 3N HCl and 50 mL n-butanol. The mixture was heated to 100° C. overnight, then additional 5 h. After cooling to RT, the solvent was removed in vacuo and the crude hydrochloride was azeotroped off butanol with hexane, then treated with 1N NaOH and extracted with CH₂Cl₂ (4×). The combined organic layers were washed with brine and dried over magnesium sulfate. Removal of the solvent in vacuo to yield crude material which was used in the next step without further purification.

### 1d) 2-Methyl-quinoline-5-carboxylic acid

To the stirring mixture of 3-aminobenzoic acid (10 g, 72.52 mmol), ferrous sulfate (5.74 g, 20.66 mmol) and sodium 3-nitrobenzene sulfonate (9.03 g, 40.11 mmol) in 150 mL of 9N HCl at 90° C., was added crotonaldehyde (10.0 mL, 121.5 mmol) over 1.5 h. After stirring for 22 h, the hot mixture was filtered and cooled to RT. The solid was precipitated out. The mixture was filtered. The solid was washed with acetone and collected to yield title product 7.009 g. LCMS: m/z 188 (M+H).

### 1e) 2-Methyl-quinoline-5-carboxylic acid (4-hydroxy-butyl)-amide

The mixture of 2-methyl-quinoline-5-carboxylic acid (250 mg, 1.34 mmol), 4-amino-1-butanol (120 mg, 1.34 mmol), EDC (260 mg, 1.34 mmol) and HOBT (18 mg, 0.13 mmol) in 10 mL of CHCl₃ was stirred at RT for 1 h. Triethylamine (0.72 mL, 5.36 mmol) was added. The resulting mixture was stirred overnight. After adding saturated NaHCO₃ and stirring for a while, the mixture was extracted with CH₂Cl₂ twice. The combined organic phase was washed with water and brine and dried over Na₂SO₄. The solvent was removed in vacuo to yield the crude product 264 mg which was used in the next step without further purification.

### 1f) 2-Methyl-quinoline-5-carboxylic acid (4-oxo-butyl)-amide

To the suspension of the crude 2-methyl-quinoline-5-carboxylic acid (4-hydroxy-butyl)-amide (264 mg, 1.02 mmol) in 10 mL of CH₂Cl₂ was added 4-methylmorpholine N-oxide (179 mg, 1.53 mmol), followed by TPAP (18 mg, 0.051 mmol) and 4 Å molecular sieves (0.5 g, 0.5 g/mmol). The mixture was stirred at RT for 1.5 h. The solvent was removed in vacuo. The residue was filtered through silica pad, eluted with 100% EtOAc, followed by ethanol and dichloromethane (1:1, v/v) to give the crude material 186 mg. This material was used in the next step without further purification.

### 1g) 2-Methyl-quinoline-5-carboxylic acid{-4-[7-(2-methyl-propanoyl)-1,2,4,5-tetrahydro-3H-3-benzazepin-3-yl]-butyl}-amide

To the mixture of crude 2-methyl-quinoline-5-carboxylic acid (4-oxo-butyl)-amide (93 mg, 0.37 mmol) and 2-methyl-1-(2,3,4,5-tetrahydro-1H-3H-3-benzazepin-7-yl)-propan-1-one (79 mg, 0.36 mmol) in 10 mL of dichloroethane was added sodium triacetoxyborohydride (150 mg, 0.73 mmol). After stirring overnight, the mixture was quenched with water. The organic layer was isolated and concentrated to yield the crude product. Purification upon Gilson HPLC, eluting with acetonitrile/water/0.1% TFA (5/95, v/v to 60/40, v/v, over 10 min), gave the desired product. LCMS: m/z 458 (M+H).

### Example 2

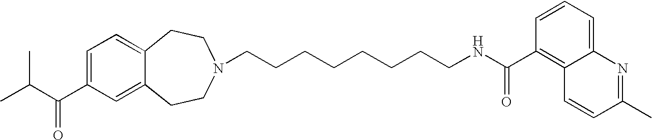

### trans-2-Methyl-quinoline-5-carboxylic acid{8-[7-(2-methyl-propanoyl)-1,2,4,5-tetrahydro-3H-3-benzazepin-3-yl]-octyl}-amide

### 2a) {8-[7-(2-Methyl-propanoyl)-1,2,4,5-tetrahydro-3H-3-benzazepin-3-yl]-octyl}-carbamic acid tert-butyl ester

To a solution of 2-methyl-1-(2,3,4,5-tetrahydro-1H-3H-3-benzazepin-7-yl)-propan-1-one (171 mg, 0.79 mmol) in 10 mL of dichloroethane, (8-oxo-octyl)-carbamic acid tert-butyl ester (192 mg, 0.79 mmol) and sodium triacetoxyborohydride (219 mg, 1.18 mmol) were added. The mixture was stirred at RT overnight, followed by at 60° C. for additional 4 h. The mixture was cooled to RT, quenched with water and concentrated. The residue was dissolved in ethyl acetate, washed with water (3×) and brine, dried over magnesium sulfate and removed the solvent in vacuo. The resulting crude was purified via column chromatography on silica gel using methanol:dichloromethane (5:95, v/v) as mobile phase to give 184 mg (53%) of the title compound. LCMS m/z 445 (M+H).

### 2b) 1-[3-(8-Amino-octyl)-2,3,4,5-tetrahydro-1H-3H-3-benzazepin-7-yl]-2-methyl-propan-1-one hydrochloride

To a solution of {8-[7-(2-methyl-propanoyl)-1,2,4,5-tetrahydro-3H-3-benzazepin-3-yl]-octyl}-carbamic acid tert-butyl ester (184 mg, 0.42 mmol) in 30 mL of ethyl acetate, was bubbled in HCl gas for 5 min. The mixture was then stirred at RT for 3 h. Removed solvent in vacuo and pumped the residue to yield the crude product. This was used in the next step without further purition.

### 2c) 2-Methyl-quinoline-5-carboxylic acid{8-[7-(2-methyl-propanoyl)-1,2,4,5-tetrahydro-3H-3-benzazepin-3-yl]-octyl}-amide

To a solution of 1-[3-(8-amino-octyl)-2,3,4,5-tetrahydro-1H-3H-3-benzazepin-7-yl]-2-methyl-propan-1-one hydrochloride (184 mg, 0.42 mmol) in 30 mL of chloroform was added DIEA (361 μL, 2.1 mmol), followed by 2-methyl-quinoline-5-carboxylic acid (116 mg, 0.62 mmol), EDC (79 mg, 0.42 mmol) and HOBT (6 mg, 0.042 mmol). The mixture was stirred at RT for 4 h. The mixture was diluted with ethyl acetate, washed with water (2×) and brine, and dried over Na₂SO₄. The solvent was removed in vacuo to yield the crude product. Purification upon Gilson HPLC, eluting with acetonitrile/water/0.1% TFA (10/90, v/v to 50/50, v/v, over 10 min), gave the desired product 48 mg. LCMS: m/z 514 (M+H).

### Example 3

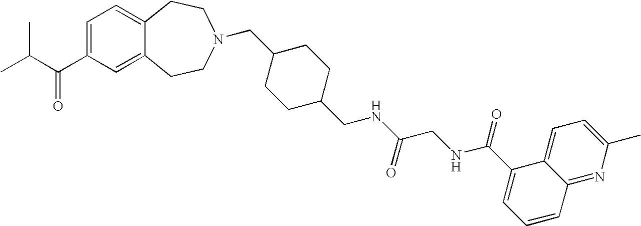

### trans-2-Methyl-quinoline-5-carboxylic acid[({4-[7-(2-methyl-propanoyl)-1,2,4,5-tetrahydro-3H-3-benzazepin-3-ylmethyl]-cyclohexylmethyl}-carbamoyl)-methyl]-amide

### 3a) {4-[7-(2-Methyl-propanoyl)-1,2,4,5-tetrahydro-3H-3-benzazepin-3-ylmethyl]-cyclohexylmethyl}-carbamic acid tert-butyl ester

Following the general procedure described in Example 2a, 2-methyl-1-(2,3,4,5-tetrahydro-1H-3H-3-benzazepin-7-yl)-propan-1-one (294 mg, 1.35 mmol) and (4-formyl-cyclohexylmethyl)-carbamic acid tert-butyl ester (326 mg, 1.35 mmol) were reacted in the presence of sodium triacetoxyborohydride (429 mg, 2.03 mmol) to give the title compound 306 mg. LCMS m/z 443 (M+H).

### 3b) 1-[3-(4-Aminomethyl-cyclohexylmethyl)-2,3,4,5-tetrahydro-1H-3H-3-benzazepin-7-yl]-2-methyl-propan-1-one

Following the general procedure described in Example 2b, {4-[7-(2-methyl-propanoyl)-1,2,4,5-tetrahydro-3H-3-benzazepin-3-ylmethyl]-cyclohexylmethyl}-carbamic acid tert-butyl ester (306 mg, 0.69 mmol) was bubbled HCl(g) to give the title compound 284 mg. LCMS m/z 343 (M+H).

### 3c) [({4-[7-(2-Methyl-propanoyl)-1,2,4,5-tetrahydro-3H-3-benzazepin-3-ylmethyl]-cyclohexylmethyl}-carbamoyl)-methyl]-carbamic acid tert-butyl ester

Following the general procedure described in Example 2c, 1-[3-(4-aminomethyl-cyclohexylmethyl)-2,3,4,5-tetrahydro-1H-3H-3-benzazepin-7-yl]-2-methyl-propan-1-one hydrochloride (95 mg, 0.24 mmol) was coupled with tert-butoxycarbonylamino-acetic acid (41 mg, 0.24 mmol) to give the crude product. This material was used in the next step without further purification. LCMS m/z 500 (M+H).

### 3d) 2-Methyl-quinoline-5-carboxylic acid [({4-[7-(2-methyl-propanoyl)-1,2,4,5-tetrahydro-3H-3-benzazepin-3-ylmethyl]-cyclohexylmethyl}-carbamoyl)-methyl]-amide

To the solution of the crude [({4-[7-(2-Methyl-propanoyl)-1,2,4,5-tetrahydro-3H-3-benzazepin-3-ylmethyl]-cyclohexylmethyl}-carbamoyl)-methyl]-carbamic acid tert-butyl ester (0.24 mmol) in 20 mL of chloroform was added 0.11 mL of TFA. The mixture was stirred at RT overnight. Additional 1 mL of TFA was added to the mixture, and continued to stirred for another 6 hr. The solvent was evaporated to give the crude 2-amino-N-{4-[7-(2-methyl-propanoyl)-1,2,4,5-tetrahydro-3H-3-benzazepin-3-ylmethyl]-cyclohexylmethyl}-acetamide. This material was coupled with 2-methyl-quinoline-5-carboxylic acid (48 mg, 0.26 mmol) by following the general procedure described in Example 2c to give the title compound 72 mg. LCMS m/z 569 (M+H).

### Example 4

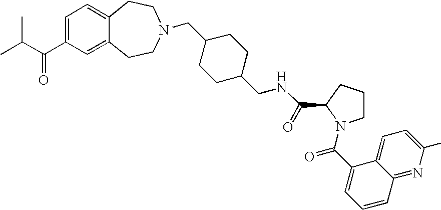

### trans-(R)-1-[1-(2-Methyl-quinolin-5-yl)-methanoyl)-pyrrolidine-2-carboxylic acid{4-[7-(2-methyl-Propanoyl)-1,2,4,5-tetrahydro-3H-3-benzazepin-3-ylmethyl-cyclohexylmethyl-amide

### 4a) (R)-2-({4-[7-(2-Methyl-propanoyl)-1,2,4,5-tetrahydro-3H-3-benzazepin-3-ylmethyl]-cyclohexylmethyl]-carbamoyl)-pyrrolidine-1-carboxylic acid tert-butyl ester

Following the general procedure described in Example 2c, 1-[3-(4-aminomethyl-cyclohexylmethyl)-2,3,4,5-tetrahydro-1H-3H-3-benzazepin-7-yl]-2-methyl-propan-1-one hydrochloride (95 mg, 0.24 mmol) was coupled with (R)-pyrrolidine-1,2-dicarboxylic acid 1-tert-butyl ester (51 mg, 0.24 mmol) to give the crude product. This material was used in the next step without further purification. LCMS m/z 540 (M+H).

### 4b) (R)-1-[1-(2-Methyl-quinolin-5-yl)-methanoyl)-pyrrolidine-2-carboxylic acid{4-[7-(2-methyl-Propanoyl)-1,2,4,5-tetrahydro-3H-3-benzazepin-3-ylmethyl]-cyclohexylmethyl]-amide

Following the general procedure described in Example 3d, the crude (R)-2-({4-[7-(2-Methyl-propanoyl)-1,2,4,5-tetrahydro-3H-3-benzazepin-3-ylmethyl]-cyclohexylmethyl}-carbamoyl)-pyrrolidine-1-carboxylic acid tert-butyl ester (0.24 mmol) was stirred with 1.11 mL of TFA to remove Boc. Then coupling with 2-methyl-quinoline-5-carboxylic acid (48 mg, 0.26 mmol) to give the title compound 65 mg. LCMS m/z 609 (M+H).

### Example 5

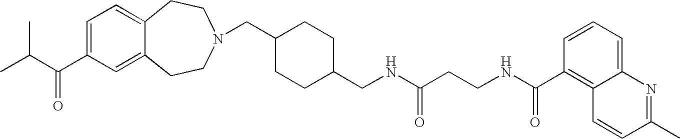

### trans-2-Methyl-quinoline-5-carboxylic acid [2-({4-[7-(2-methyl-propanoyl)-1,2,4,5-tetrahydro-3H-3-benzazepin-3-ylmethyl]-cyclohexylmethyl]-carbamoyl)-ethyl]-amide

### 5a) [2-({4-[7-(2-Methyl-propanoyl)-1,2,4,5-tetrahydro-3H-3-benzazepin-3-yl]-cyclohexylmethyl}-carbamoyl)-ethyl]-carbamic acid tert-butyl ester

Following the general procedure described in Example 2c, 1-[3-(4-aminomethyl-cyclohexylmethyl)-2,3,4,5-tetrahydro-1H-3H-3-benzazepin-7-yl]-2-methyl-propan-1-one hydrochloride (95 mg, 0.24 mmol) was coupled with 3-tert-butoxycarbonylamino-propionic acid (45 mg, 0.24 mmol) to give the crude product. This material was used in the next step without further purification. LCMS m/z 514 (M+H).

### 5b) 2-Methyl-quinoline-5-carboxylic acid [2-({4-[7-(2-methyl-propanoyl)-1,2,4,5-tetrahydro-3H-3-benzazepin-3-ylmethyl]-cyclohexylmethyl}-carbamoyl)-ethyl]-amide

Following the general procedure described in Example 4d, the crude [2-({4-[7-(2-methyl-propanoyl)-1,2,4,5-tetrahydro-3H-3-benzazepin-3-yl]-cyclohexyl-methyl}-carbamoyl)-ethyl]-carbamic acid tert-butyl ester (0.24 mmol) was stirred with 1.11 mL of TFA to remove the Boc protecting group. The resulting crude amine was then coupled with 2-methyl-quinoline-5-carboxylic acid (48 mg, 0.26 mmol) to give the title compound 68 mg. LCMS m/z 583 (M+H).

### Example 6

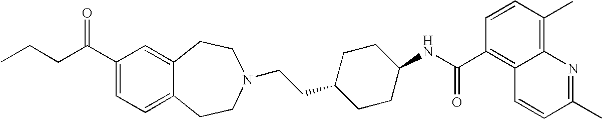

### trans-2,8-Dimethyl-quinoline-5-carboxylic acid{-4-[2-(7-butyryl-1,2,4,5-tetrahydro-3H-3-benzazepin-3-yl)-ethyl]-cyclohexyl}-amide

### 6a) 1-(7-Butyryl-1,2,4,5-tetrahydro-3H-3-benzazepin-3-yl)-2,2,2-trifluoro-ethanone

2,2,2-trifluoro-1-(1,2,4,5-tetrahydro-3H-3-benzazepin-3-yl)-ethanone (Example 1a) (1.41 g, 5.80 mmol) ws dissolved in 7 mL carbondisulfide. Aluminum chloride (4.0 g, 17.4 mmol) was added and the mixture was heated to 45° C. Butyryl chloride (1.8 mL, 17.4 mmol) was added dropwise over 15 min. Stirring was continued at 45° C. for 1 h and then at RT for 1 h. The reaction was quenched with 6N HCl, and diluted with water and ethyl acetate. The aquous layer was extracted with 2×10 mL EtOAc and the combined organic layers were washed with saturated aqueous sodium bicarbonate and brine and dried over magnesium sulfate. The solvent was removed in vacuo and the crude material was recrystallized from EtOAc/hexanes to give 1.44 g of an off-white solid. LCMS: m/z 314(M+H).

### 6b) 1-(2,3,4,5-Tetrahydro-1H-3H-3-benzazepin-7-yl)-butan-1-one

1-(7-Butyryl-1,2,4,5-tetrahydro-3H-3-benzazepin-3-yl)-2,2,2-trifluoroethanone (1.44 g, 4.60 mmol) was dissolved in 25 mL 3N HCl and 15 mL n-butanol. The mixture was heated to reflux for 6 h. After cooling to RT the solvent was removed in vacuo and the crude hydrochloride was treated with 2N NaOH and extracted with 2×100 mL EtOAc. The combined organic layers were washed with brine and dried over magnesium sulfate. Removal of the solvent in vacuo and recrystallization from EtOAC/hexanes yielded 0.85 g of a white solid. LCMS: m/z 218(M+H).

### 6c) 1-{3-[2-(4-Amino-cyclohexyl)-ethyl]-2,3,4,5-tetrahydro-1H-3H-3-benzazepin-7-yl}-butan-1-one bistrifluoroacetate

To a solution of 1-(2,3,4,5-tetrahydro-1H-3H-3-benzazepin-7-yl)-butan-1-one (113 mg, 0.47 mmol) and [4-(2-oxo-ethyl)-cyclohexyl]-carbamic acid tert-butyl ester (102 mg, 0.47 mmol) in 5.8 mL of dichloromethane, sodium triacetoxyborohydride (149 mg, 0.71 mmol) was added. The mixture was stirred at RT for 14 h. The mixture was diluted with dichloromethane and washed with 10% aq. Na₂CO₃. The aqueous phase was extracted with dichloromethane (40 mL). The combined organic phases was washed with brine, dried over magnesium sulfate and removed the solvent in vacuo. The resulting crude was dissolved in the mixture of 10 mL of dichloromethane and 4 mL of trifluoroacetic acid. The resulting mixture was heated to 60° C. for 1 h, and then cooled to RT. The solvent was removed in vacuo to yield 230 mg (86%) of the title compound: LCMS m/z 343 (M+H).

### 6d) 2,8-Dimethyl-quinoline-5-carboxylic acid{-4-[2-(7-butyryl-1,2,4,5-tetrahydro-3H-3-benzazepin-3-yl)-ethyl]-cyclohexyl}-amide

The mixture of 1-{3-[2-(4-amino-cyclohexyl)-ethyl]-2,3,4,5-tetrahydro-1H-3H-3-benzazepin-7-yl}-butan-1-one bistrifluoroacetate (58 mg, 0.1 mmol), 2,8-dimethyl-quinoline-5-carboxylic acid (51 mg, 0.25 mmol), EDC (48 mg, 0.25 mmol) and DIEA (44 μL, 0.25 mmol) in 1 mL of CH₂Cl₂ was stirred at RT for 1 h. The solvent was removed in vacuo to yield the crude product. Purification upon Gilson HPLC, eluting with acetonitrile/water/0.5% TFA (10/90, v/v to 90/10, v/v, over 10 min), gave the desired product 6 mg (9%). LCMS: m/z 526 (M+H).

### Example 7

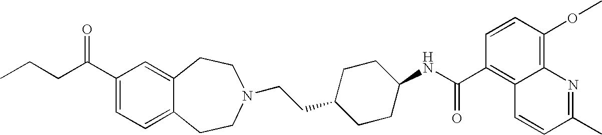

### trans-8-Methoxy-2-methyl-quinoline-5-carboxylic acid{4-[2-(7-butyryl-1,2,4,5-tetrahydro-3H-3-benzazepin3-yl)-ethyl]-cyclohexyl}-amide

Following the general procedure outlined in Example 6d, 1-{3-[2-(4-amino-cyclohexyl)-ethyl]-2,3,4,5-tetrahydro-1H-3H-3-benzazepin-7-yl}-butan-1-one bistrifluoroacetate (58 mg, 0.1 mmol) coulped with 8-methoxy-2-methyl-quinoline-5-carboxylic acid (55 mg, 0.25 mmol) to yield the title compound 17 mg (25%). LCMS m/z 542 (M+H).

### Example 8

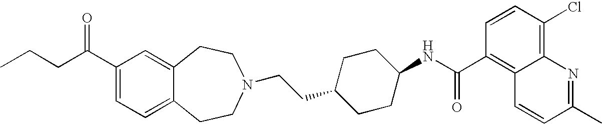

### trans-8-Chloro-2-methyl-quinoline-5-carboxylic acid{4-[2-(7-butyryl-1,2,4,5-tetrahydro-3H-3-benzazepin3-yl)-ethyl]-cyclohexyl}-amide

Following the general procedure outlined in Example 6d, 1-{3-[2-(4-amino-cyclohexyl)-ethyl]-2,3,4,5-tetrahydro-1H-3H-3-benzazepin-7-yl}-butan-1-one bistrifluoroacetate (58 mg, 0.1 mmol) coulped with 8-chloro-2-methyl-quinoline-5-carboxylic acid (56 mg, 0.25 mmol) to yield the title compound 6 mg (9%). LCMS m/z 546 (M+H).

### Example 9

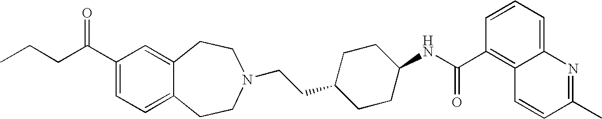

### trans-2-Methyl-quinoline-5-carboxylic acid{-4-[2-(7-butyryl-1,2,4,5-tetrahydro-3H-3-benzazepin3-yl)-ethyl]-cyclohexyl}-amide

Following the general procedure outlined in Example 6d, 1-{3-[2-(4-amino-cyclohexyl)-ethyl]-2,3,4,5-tetrahydro-1H-3H-3-benzazepin-7-yl}-butan-1-one bistrifluoroacetate (58 mg, 0.1 mmol) coulped with 2-methyl-quinoline-5-carboxylic acid (48 mg, 0.25 mmol) to yield the title compound 16 mg (26%). LCMS m/z 512 (M+H).

### Example 10

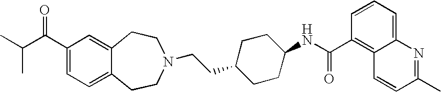

### trans-2-Methyl-quinoline-5-carboxylic acid{-4-[2-(7-(2-methyl-propanoyl)-1,2,4,5-tetrahydro-3H-3-benzazepin3-yl)-ethyl]-cyclohexyl}-amide

### 10a) 1-{3-[2-(4-Amino-cyclohexyl)-ethyl]-2,3,4,5-tetrahydro-1H-3H-3-benzazepin-7-yl}-2-methylpropan-1-one

Following the general procedure of example 6c, 2-methyl-1-(2,3,4,5-tetrahydro-1H-3H-3-benzazepin-7-yl)-propan-1-one (2.7 g, 12.4 mmol) and [4-(2-oxo-ethyl)-cyclohexyl]-carbamic acid tert-butyl ester (3.0 mg, 12.4 mmol) in 120 mL dichloroethane was treated with sodium triacetoxyborohydride (3.9 g, 18.6 mmol). The crude product (5.0 g) was dissolved in 75 mL dichloromethane and TFA (7.5 mL) was added. The mixture was stirred at room temperature for 2 h after which the solvent was evaporated. The crude product was dissolved in a mixture of dichloromethane and ethyl acetate and washed with 2×100 mL 10% aqueous sodium carbonate and brine. The aqueous layer was extracted with 2×100 mL ethyl acetate and the combined organic layers were dried over magnesium sulfate. Following filtration, the solvent was evaporated in vacuo to give 2.9 g (68%) of the title compound. LCMS m/z 343 (M+H).

### 10b) trans-2-Methyl-quinoline-5-carboxylic acid{-4-[2-(7-(2-methyl-propanoyl)-1,2,4,5-tetrahydro-3H-3-benzazepin3-yl)-ethyl]-cyclohexyl}-amide hydrochloride

1-{3-[2-(4-Amino-cyclohexyl)-ethyl]-2,3,4,5-tetrahydro-1H-3H-3-benzazepin-7-yl}-2-methylpropan-1-one (275 mg, 0.80 mmol) and 2-methyl-quinoline-5-carboxylic acid (197 mg, 0.88 mmol) was dissolved/suspended in 20 mL chloroform. Diisopropylethylamine (1.0 mL, 4.0 mmol), EDC hydrochloride (153 mg, 0.80 mmol) and HOBt (11 mg, 0.08 mmol) was added and the reaction mixture was stirred at room temp. for 14 h. Dichloromethane was added and the mixture was washed with 2×70 mL 10% aqueous potassium carbonate, brine and dried over magnesium sulfate. The solvent was evaporated in vacuo and the crude material was dissolved in dichloromethane. 4N HCl in dioxane (0.2 ml, 0.8 mmol) was added. Diethyl ether was added and the resulting precipitate was filtered and washed with diethyl ether yielding 320 mg of the title compound. LCMS m/z 512 (M+H)

### Example 11

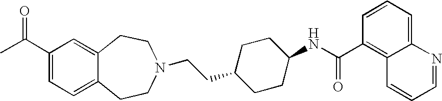

### trans-7-Acetyl-3-(2-(1-(4-(5-quinolinyl)carboxamido)cyclohexyl)ethyl)-2,3,4,5-tetrahydro-1H-3-benzazepine

A mixture of 7-acetyl-trans-3-(2-(1-(4-amino)cyclohexyl)ethyl)-2,3,4,5-tetrahydro-1H-3-benzazepine (0.105 g, 0.334 mmol) (prepared according to WO 00/21951), quinoline-5-carboxylic acid (0.064 g, 0.368 mmol), 1-(3-dimethylaminopropyl)-3-ethylcarbodiimide hydrochloride (0.071 g, 0.368 mmol) and 1-hydroxybenzotriazole hydrate (0.01 g, 0.065 mmol) in dichloromethane (6 ml) was shaken for 18 h. Saturated aqueous sodium hydrogen carbonate (6 ml) was added and shaking continued for a further 0.5 h. The organic layer was separated and pipetted onto a column of silica (10 g). Elution with 30-100% ethyl acetate-hexane gradient then 1-10% methanol-ethyl acetate gradient gave the title compound as a colorless solid (1 g, 64%). Mass spectrum (API⁺): 470 (MH⁺);

### Examples 12-81

Examples 12-81 were prepared following the general procedure of example 11:

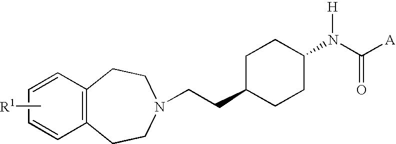

| | | | Characterising Data: |
|---|---|---|---|
| | | | Mass Spectrum |
| Example | R¹ | A | (API⁺) |
| 12 | 7-COMe | —CH₂C₆H₄(4-F) | Mass: 451 (MH⁺) |
| 13 | 7-COMe | —C₆H₄(3-(3-(5-methyl)- | Mass: 501 (MH⁺) |
| | | 1,2,4-oxadiazolyl)) | |
| 14 | 7-COMe | 3-pyrrolo[2,3-b]pyridyl | Mass: 459 (MH⁺) |
| 15 | 7-COMe | trans-CH═CHC₆H₄(4-F) | Mass: 463 (MH⁺) |
| 16 | 7-COMe | trans-CH═CHC₆H₄(3-OMe) | Mass: 475 (MH⁺) |
| 17 | 7-COMe | trans-CH═CHC₆H₄(2-CN) | Mass: 470 (MH⁺) |
| 18 | 7-COMe | trans-CH═CH(3-thiophenyl) | Mass: 451 (MH⁺) |
| 19 | 7-COMe | trans-CH═CH(8-(1,2- | Mass: 512 (MH⁺) |
| | | dihydro-2-oxo)-quinolinyl) | |
| 20 | 7-COMe | CH₂(3-benzothiophenyl) | Mass: 489 (MH⁺) |
| 21 | 7-COMe | trans-CH═CC₆H₄(4- | Mass: 502 (MH⁺) |
| | | NHCOMe) | |
| 22 | 7-COMe | —CH₂(6-(2-amino)- | Mass: 505 (MH⁺) |
| | | benzothiazolyl) | |
| 23 | 7-COMe | 8-(1,4-dihydro-4-oxo)- | Mass: 486 (MH⁺) |
| | | quinolinyl | |
| 24 | 7-COMe | trans-CH═CHC₆H₄(2- | Mass: 487 (MH⁺) |
| | | COMe) | |
| 25 | 7-COMe | —CH₂(2-benzothiophenyl) | Mass: 489 (MH⁺) |
| 26 | 7-COCH(CH₃)₂ | —(CH₂)₃NH₂ | Mass: 428 (MH⁺) |
| 27 | 7-COCH(CH₃)₂ | 2-pyrazine | Mass: 449 (MH⁺) |
| 28 | 7-COCH(CH₃)₂ | —CH₂(4-pyridinyl) | Mass: 462 (MH⁺) |
| 29 | 7-COCH(CH₃)₂ | trans-CH═CH-(1H-imidazol- | Mass: 463 (MH⁺) |
| | | 4-yl) | |
| 30 | 7-COCH(CH₃)₂ | (R)-CH(NH₂)C₆H₅ | Mass: 476 (MH⁺) |
| 31 | 7-COCH(CH₃)₂ | 2-indole | Mass: 486 (MH⁺) |
| 32 | 7-COCH(CH₃)₂ | CH₂—CH═CH—C₆H₅ | Mass: 487 (MH⁺) |
| 33 | 7-COCH(CH₃)₂ | —C₆H₄(2-NMe₂) | Mass: 490 (MH⁺) |
| 34 | 7-COCH(CH₃)₂ | —C₆H₄(3-NMe₂) | Mass: 490 (MH⁺) |
| 35 | 7-COCH(CH₃)₂ | (R)-CH(NH₂)(3-pyridinyl) | Mass: 591 (MH⁺) |
| 36 | 7-COCH(CH₃)₂ | 4-pyridinyl | Mass: 448 (MH⁺) |
| 37 | 7-COCH(CH₃)₂ | 2-quinoline | Mass: 498 (MH⁺) |
| 38 | 7-COCH(CH₃)₂ | 5-quinoxaline | Mass: 499 (MH⁺) |
| 39 | 7-COCH(CH₃)₂ | 2-(1,8-naphthyridine | Mass: 499 (MH⁺) |
| 40 | 7-COCH(CH₃)₂ | 2-(1,6-naphthyridine | Mass: 499 (MH⁺) |
| 41 | 7-COCH(CH₃)₂ | —CH₂(3-indole) | Mass: 500 (MH⁺) |
| 42 | 7-COCH(CH₃)₂ | 2-(1-methylindole) | Mass: 500 (MH⁺) |
| 43 | 7-COCH(CH₃)₂ | —CH═CH—CO—C₆H₅ | Mass: 501 (MH⁺) |
| 44 | 7-COCH(CH₃)₂ | —CH₂C₆H₄(4-NMe₂) | Mass: 504 (MH⁺) |
| 45 | 7-COCH(CH₃)₂ | —CH₂SCH₂C₆H₅ | Mass: 507 (MH⁺) |
| 46 | 7-COCH(CH₃)₂ | 3-(pyridine oxide) | Mass: 464 (MH⁺) |
| 47 | 7-COCH(CH₃)₂ | —C₆H₄(2-(1H-pyrrol-1-yl)) | Mass: 512 (MH⁺) |
| 48 | 7-COCH(CH₃)₂ | —C₆H₄(4-(1H-pyrrol-1-yl)) | Mass: 512 (MH⁺) |
| 49 | 7-COCH(CH₃)₂ | 5-pyridinyl-(2-(1H-pyrrol-1- | Mass: 513 (MH⁺) |
| | | yl)) | |
| 50 | 7-COCH(CH₃)₂ | —(CH₂)₂-3-indole | Mass: 514 (MH⁺) |
| 51 | 7-COCH(CH₃)₂ | 1-phenyl | Mass: 515 (MH⁺) |
| | | cyclopentane-1-yl | |
| 52 | 7-COCH(CH₃)₂ | —CH₂(1-(5-hydroxy)-1H- | Mass: 517 (MH⁺) |
| | | benzimidazol) | |
| 53 | 7-COCH(CH₃)₂ | —(CH₂)₂C₆H₄(4-OMe) | Mass: 519 (MH⁺) |
| 54 | 7-COCH(CH₃)₂ | -(R)-CH(NH₂)CH₂OCH₂ | Mass: 520 (MH⁺) |
| | | C₆H₅ | |
| 55 | 7-COCH(CH₃)₂ | -5-(2,8-dimethylquinoline) | Mass: 526 (MH⁺) |
| 56 | 7-COCH(CH₃)₂ | -4-(5-methyl-2-phenyl-2H- | Mass: 528 (MH⁺) |
| | | 1,2,3-triazole) | |
| 57 | 7-COCH(CH₃)₂ | —(CH₂)₃NHCO₂-t-Bu | Mass: 528 (MH⁺) |
| 58 | 7-COCH(CH₃)₂ | -4-(2-(3-pyridinyl)-1,3- | Mass: 531 (MH⁺) |
| | | thiazole) | |
| 59 | 7-COCH(CH₃)₂ | 3-indole | Mass: 486 (MH⁺) |
| 60 | 7-COCH(CH₃)₂ | —(CH₂)₂—CONHCH₂C₆H₅ | Mass: 532 (MH⁺) |
| 61 | 7-COCH(CH₃)₂ | -5-(8-chloroquinoline) | Mass: 532 (MH⁺) |
| 62 | 7-COCH(CH₃)₂ | —(CH₂)₂—C₆H₄-(3,4- | Mass: 535 (MH⁺) |
| | | dimethoxy) | |
| 63 | 7-COCH(CH₃)₂ | —CH(C₆H₅)₂ | Mass: 537 (MH⁺) |
| 64 | 7-COCH(CH₃)₂ | —C₆H₄-(2-NHSO₂Me) | Mass: 540 (MH⁺) |
| 65 | 7-COCH(CH₃)₂ | —CH₂-(2-pyrimidinyl) | Mass: 495 (MH⁺) |
| 66 | 7-COCH(CH₃)₂ | -5-(2-methyl-8- | Mass: 542 (MH⁺) |
| | | methoxyquinoline) | |
| 67 | 7-COCH(CH₃)₂ | 1-isoquinoline | Mass: 498 (MH⁺) |
| 68 | 7-COCH(CH₃)₂ | —(CH₂)₂SCH₂CO₂-t-Bu | Mass: 544 (MH⁺) |
| 69 | 7-COCH(CH₃)₂ | —(CH₂)₄-NHCOC₆H₅ | Mass: 546 (MH⁺) |
| 70 | 7-COCH(CH₃)₂ | -5-(2-methyl-8- | Mass: 546 (MH⁺) |
| | | chloroquinoline) | |
| 71 | 7-COCH(CH₃)₂ | —C₆H₄-(2-CO₂-t-Bu) | Mass: 547 (MH⁺) |
| 72 | 7-COCH(CH₃)₂ | —CH₂—NHCO(3-pyridinyl) | Mass: 505 (MH⁺) |
| 73 | 7-COCH(CH₃)₂ | —CH₂CH(C₆H₅)₂ | Mass: 551 (MH⁺) |
| 74 | 7-COCH(CH₃)₂ | —CH₂-(2-naphtyl) | Mass: 511 (MH⁺) |
| 75 | 7-COCH(CH₃)₂ | (R)-CH(NHCO₂-t-Bu)C₆H₅ | Mass: 576 (MH⁺) |
| 76 | 7-COCH(CH₃)₂ | —(CH₂)₂—NHSO₂—C₆H₄(4-Me) | Mass: 568 (MH⁺) |
| 77 | 7-COCH(CH₃)₂ | (R)-CH(NHCO₂-t-Bu)CH₂- | Mass: 591 (MH⁺) |
| | | (3-pyridinyl) | |
| 78 | 7-COCH(CH₃)₂ | (R)-CH(NHCO₂-t- | Mass: 620 (MH⁺) |
| | | Bu)CH₂OCH₂C₆H₅ | |

### BIOLOGICAL EXAMPLES

The inhibitory effects of compounds at the mAChRs of the present invention are determined by the following in vitro and in vivo functional assays:

Analysis of Inhibition of Receptor Activation by Calcium Mobilization:

Stimulation of mAChRs expressed on CHO cells were analyzed by monitoring receptor-activated calcium mobilization as previously described (Sarau, H. M., R. S. Ames, J. Chambers, C. Ellis, N. Elshourbagy, J. J. Foley, D. B. Schmidt, R. M. Muccitelli, O. Jenkins, P. R. Murdock, N. C. Herrity, W. Halsey, G. Sathe, A. I. Muir, P. Nuthulaganti, G. M. Dytko, P. T. Buckley, S. Wilson, D. J. Bergsma, and D. W. Hay. 1999. Identification, molecular cloning, expression, and characterization of a cysteinyl leukotriene receptor. Mol Pharmacol 56:657-663). CHO cells stably expressing M₃ mAChRs were plated in 96 well black wall/clear bottom plates. After 18 to 24 hours, media was aspirated and replaced with 100 μl of load media (EMEM with Earl's salts, 0.1% RIA-grade BSA (Sigma, St. Louis Mo.), and 4 μM Fluo-3-acetoxymethyl ester fluorescent indicator dye (Fluo-3 AM, Molecular Probes, Eugene, Oreg.) and incubated 1 hr at 37° C. The dye-containing media was then aspirated, replaced with fresh media (without Fluo-3 AM), and cells were incubated for 10 minutes at 37° C. Cells were then washed 3 times and incubated for 10 minutes at 37° C. in 100 μl of assay buffer (0.1% gelatin (Sigma), 120 mM NaCl, 4.6 mM KCl, 1 mM KH₂ PO₄, 25 mM NaH CO₃, 1.0 mM CaCl₂, 1.1 mM MgCl₂, 11 mM glucose, 20 mM HEPES (pH 7.4)). 50 μl of compound (1×10⁻¹¹-1×10⁻⁵M final in the assay) was added and the plates were incubated for 10 min. at 37° C. Plates were then placed into a fluorescent light intensity plate reader (FLIPR, Molecular Probes) where the dye loaded cells were exposed to excitation light (488 nm) from a 6 watt argon laser. Cells were activated by adding 50 μl of acetylcholine (0.1-10 nM final), prepared in buffer containing 0.1% BSA, at a rate of 50 μl/sec. Calcium mobilization, monitored as change in cytosolic calcium concentration, was measured as change in 566 nm emission intensity. The change in emission intensity is directly related to cytosolic calcium levels (Sullivan, E., E. M. Tucker, and 1. L. Dale. 1999. Measurement of [Ca2+] using the Fluorometric Imaging Plate Reader (FLIPR). Methods Mol Biol 114:125-133). The emitted fluorescence from all 96 wells is measured simultaneously using a cooled CCD camera. Data points are collected every second. This data was then plotting and analyzed using GraphPad PRISM software.

Methacholine-Induced Bronchoconstriction

Airway responsiveness to methacholine was determined in awake, unrestrained BalbC mice (n=6 each group). Barometric plethysmography was used to measure enhanced pause (Penh), a unitless measure that has been shown to correlate with the changes in airway resistance that occur during bronchial challenge with methacholine (Hamelmann, E., J. SCHWARZE, K. TAKEDA, A. OSHIBA, G. á. LARSEN, C. á. IRVIN, and E. á. GELFAND. 1997. Noninvasive Measurement of Airway Responsiveness in Allergic Mice Using Barometric Plethysmography. Am. J. Respir. Crit. Care Med. 156:766-775). Mice were pretreated with 50 μl of compound (0.003-10 μg/mouse) in 50 μl of vehicle (10% DMSO) intranasally, i.v., i.p. or p.o, and were then placed in the plethysmography chamber. Once in the chamber, the mice were allowed to equilibrate for 10 min before taking a baseline Penh measurement for 5 minutes. Mice were then challenged with an aerosol of methacholine (10 mg/ml) for 2 minutes. Penh was recorded continuously for 7 min starting at the inception of the methacholine aerosol, and continuing for 5 minutes afterward. Data for each mouse were analyzed and plotted by using GraphPad PRISM software.

The present compounds are useful for treating a variety of indications, including but not limited to respiratory-tract disorders such as chronic obstructive lung disease, chronic bronchitis, asthma, chronic respiratory obstruction, pulmonary fibrosis, pulmonary emphysema, and allergic rhinitis; gastrointestinal-tract disorders such as irritable bowel syndrome, spasmodic colitis, gastroduodenal ulcers, gastrointestinal convulsions or hyperanakinesia, diverticulitis, pain accompanying spasms of gastrointestinal smooth musculature; urinary-tract disorders accompanying micturition disorders including neurogenic pollakisuria, neurogenic bladder, nocturnal enuresis, psychosomatic bladder, incontinence associated with bladder spasms or chronic cystitis, urinary urgency or pollakiuria, and motion sickness.

Methods of administering the present compounds will be readily apparent to the skilled artisan.

Dry powder compositions for topical delivery to the lung by inhalation may, for example, be presented in capsules and cartridges of for example gelatine, or blisters of for example laminated aluminium foil, for use in an inhaler or insufflator. Formulations generally contain a powder mix for inhalation of the compound of the invention and a suitable powder base (carrier substance) such as lactose or starch. Use of lactose is preferred. Each capsule or cartridge may generally contain between 20 μg-10 mg of the compound of formula (I) optionally in combination with another therapeutically active ingredient. Alternatively, the compound of the invention may be presented without excipients.

Suitably, the medicament dispenser is of a type selected from the group consisting of a reservoir dry powder inhaler (RDPI), a multi-dose dry powder inhaler (MDPI), and a metered dose inhaler (MDI).

By reservoir dry powder inhaler (RDPI) it is meant an inhaler having a reservoir form pack suitable for comprising multiple (un-metered doses) of medicament in dry powder form and including means for metering medicament dose from the reservoir to a delivery position. The metering means may for example comprise a metering cup, which is movable from a first position where the cup may be filled with medicament from the reservoir to a second position where the metered medicament dose is made available to the patient for inhalation.

By multi-dose dry powder inhaler (MDPI) is meant an inhaler suitable for dispensing medicament in dry powder form, wherein the medicament is comprised within a multi-dose pack containing (or otherwise carrying) multiple, define doses (or parts thereof) of medicament. In a preferred aspect, the carrier has a blister pack form, but it could also, for example, comprise a capsule-based pack form or a carrier onto which medicament has been applied by any suitable process including printing, painting and vacuum occlusion.

The formulation can be pre-metered (eg as in Diskus, see GB 2242134 or Diskhaler, see GB 2178965, 2129691 and 2169265) or metered in use (eg as in Turbuhaler, see EP 69715). An example of a unit-dose device is Rotahaler (see GB 2064336). The Diskus inhalation device comprises an elongate strip formed from a base sheet having a plurality of recesses spaced along its length and a lid sheet hermetically but peelably sealed thereto to define a plurality of containers, each container having therein an inhalable formulation containing a compound of formula (I) preferably combined with lactose. Preferably, the strip is sufficiently flexible to be wound into a roll. The lid sheet and base sheet will preferably have leading end portions which are not sealed to one another and at least one of the said leading end portions is constructed to be attached to a winding means. Also, preferably the hermetic seal between the base and lid sheets extends over their whole width. The lid sheet may preferably be peeled from the base sheet in a longitudinal direction from a first end of the said base sheet.

In one aspect, the multi-dose pack is a blister pack comprising multiple blisters for containment of medicament in dry powder form. The blisters are typically arranged in regular fashion for ease of release of medicament therefrom.

In one aspect, the multi-dose blister pack comprises plural blisters arranged in generally circular fashion on a disc-form blister pack. In another aspect, the multi-dose blister pack is elongate in form, for example comprising a strip or a tape.

Preferably, the multi-dose blister pack is defined between two members peelably secured to one another. U.S. Pat. Nos. 5,860,419, 5,873,360 and 5,590,645 describe medicament packs of this general type. In this aspect, the device is usually provided with an opening station comprising peeling means for peeling the members apart to access each medicament dose. Suitably, the device is adapted for use where the peelable members are elongate sheets which define a plurality of medicament containers spaced along the length thereof, the device being provided with indexing means for indexing each container in turn. More preferably, the device is adapted for use where one of the sheets is a base sheet having a plurality of pockets therein, and the other of the sheets is a lid sheet, each pocket and the adjacent part of the lid sheet defining a respective one of the containers, the device comprising driving means for pulling the lid sheet and base sheet apart at the opening station.

By metered dose inhaler (MDI) it is meant a medicament dispenser suitable for dispensing medicament in aerosol form, wherein the medicament is comprised in an aerosol container suitable for containing a propellant-based aerosol medicament formulation. The aerosol container is typically provided with a metering valve, for example a slide valve, for release of the aerosol form medicament formulation to the patient. The aerosol container is generally designed to deliver a predetermined dose of medicament upon each actuation by means of the valve, which can be opened either by depressing the valve while the container is held stationary or by depressing the container while the valve is held stationary.

Where the medicament container is an aerosol container, the valve typically comprises a valve body having an inlet port through which a medicament aerosol formulation may enter said valve body, an outlet port through which the aerosol may exit the valve body and an open/close mechanism by means of which flow through said outlet port is controllable.

The valve may be a slide valve wherein the open/close mechanism comprises a sealing ring and receivable by the sealing ring a valve stem having a dispensing passage, the valve stem being slidably movable within the ring from a valve-closed to a valve-open position in which the interior of the valve body is in communication with the exterior of the valve body via the dispensing passage.

Typically, the valve is a metering valve. The metering volumes are typically from 10 to 100 μl, such as 25 μl, 50 μl or 63 μl. Suitably, the valve body defines a metering chamber for metering an amount of medicament formulation and an open/close mechanism by means of which the flow through the inlet port to the metering chamber is controllable. Preferably, the valve body has a sampling chamber in communication with the metering chamber via a second inlet port, said inlet port being controllable by means of an open/close mechanism thereby regulating the flow of medicament formulation into the metering chamber.

The valve may also comprise a ‘free flow aerosol valve’ having a chamber and a valve stem extending into the chamber and movable relative to the chamber between dispensing and non-dispensing positions. The valve stem has a configuration and the chamber has an internal configuration such that a metered volume is defined therebetween and such that during movement between is non-dispensing and dispensing positions the valve stem sequentially: (i) allows free flow of aerosol formulation into the chamber, (ii) defines a closed metered volume for pressurized aerosol formulation between the external surface of the valve stem and internal surface of the chamber, and (iii) moves with the closed metered volume within the chamber without decreasing the volume of the closed metered volume until the metered volume communicates with an outlet passage thereby allowing dispensing of the metered volume of pressurized aerosol formulation. A valve of this type is described in U.S. Pat. No. 5,772,085. Additionally, intra-nasal delivery of the present compounds is effective.

To formulate an effective pharmaceutical nasal composition, the medicament must be delivered readily to all portions of the nasal cavities (the target tissues) where it performs its pharmacological function. Additionally, the medicament should remain in contact with the target tissues for relatively long periods of time. The longer the medicament remains in contact with the target tissues, the medicament must be capable of resisting those forces in the nasal passages that function to remove particles from the nose. Such forces, referred to as ‘mucociliary clearance’, are recognised as being extremely effective in removing particles from the nose in a rapid manner, for example, within 10-30 minutes from the time the particles enter the nose.

Other desired characteristics of a nasal composition are that it must not contain ingredients which cause the user discomfort, that it has satisfactory stability and shelf-life properties, and that it does not include constituents that are considered to be detrimental to the environment, for example ozone depletors.

A suitable dosing regime for the formulation of the present invention when administered to the nose would be for the patient to inhale deeply subsequent to the nasal cavity being cleared. During inhalation the formulation would be applied to one nostril while the other is manually compressed. This procedure would then be repeated for the other nostril.

A preferable means for applying the formulation of the present invention to the nasal passages is by use of a pre-compression pump. Most preferably, the pre-compression pump will be a VP7 model manufactured by Valois SA. Such a pump is beneficial as it will ensure that the formulation is not released until a sufficient force has been applied, otherwise smaller doses may be applied. Another advantage of the pre-compression pump is that atomisation of the spray is ensured as it will not release the formulation until the threshold pressure for effectively atomising the spray has been achieved. Typically, the VP7 model may be used with a bottle capable of holding 10-50 ml of a formulation. Each spray will typically deliver 50-100 μl of such a formulation, therefore, the VP7 model is capable of providing at least 100 metered doses.

### EXAMPLES OF NASAL FORMULATIONS

### Example 1

### Nasal Formulation Containing Active

A formulation for intranasal delivery was prepared with ingredients as follows:

| | | |
|---|---|---|
| | to 100% | |
| Active | 0.1% | w/w |
| Polysorbate 80 | 0.025% | w/w |
| Avicel RC591 | 1.5% | w/w |
| Dextrose | 5.0% | w/w |
| BKC | 0.015% | w/w |
| EDTA | 0.015% | w/w |
| water | to 100% | |

in a total amount suitable for 120 actuations and the formulation was filled into a bottle fitted with a metering valve adapted to dispense 50 or 100 μl per actuation. The device was fitted into a nasal actuator (Valois).

### Example 2

### Nasal Formulation Containing Active

A formulation for intranasal delivery was prepared with ingredients as follows:

| | | |
|---|---|---|
| Active | 0.005% | w/w |
| Tyloxapol | 2% | w/w |
| dextrose | 5% | w/w |
| BKC | 0.015% | w/w |
| EDTA | 0.015% | w/w |
| water | to 100% | |

in a total amount suitable for 120 actuations and the formulation was filled into a bottle (plastic or glass) fitted with a metering valve adapted to dispense 50 or 100 μl per actuation
The device was fitted into a nasal actuator (Valois, e.g. VP3, VP7 or VP7D)

### Example 3

### Nasal Formulation Containing Active

A formulation for intranasal delivery was prepared with ingredients as follows:

| | | |
|---|---|---|
| active | 0.05% | w/w |
| Triton X-100 | 5% | w/w |
| Dextrose | 4% | w/w |
| BKC | 0.015% | w/w |
| EDTA | 0.015% | w/w |
| water | to 100% | |

in a total amount suitable for 120 actuations and the formulation was filled into a bottle fitted with a metering valve adapted to dispense 50 or 100 μl per actuation.

### Example 4

### Nasal Formulation Containing Active

A formulation for intranasal delivery was prepared with ingredients as follows:

| | | |
|---|---|---|
| active | 0.05% | w/w |
| Tyloxapol | 5% | w/w |
| dextrose | 5% | w/w |
| BKC | 0.015% | w/w |
| EDTA | 0.015% | w/w |
| water | to 100% | |

in a total amount suitable for 120 actuations and the formulation was filled into a bottle fitted with a metering valve adapted to dispense 50 or 100 μl per actuation The device was fitted into a nasal actuator (Valois).

Throughout the specification and the claims which follow, unless the context requires otherwise, the word ‘comprise’, and variations such as ‘comprises’ and ‘comprising’, will be understood to imply the inclusion of a stated integer or step or group of integers but not to the exclusion of any other integer or step or group of integers or steps.

What is claimed is:

1. A compound according to Formula I herein below: 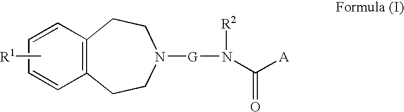
wherein:
R¹ is selected from the group consisting of a substituent selected from: C₁₋₆ alkanoyl, aroyl and aroylC₁₋₆alkyl, all optionally substituted;
R² is selected from the group consisting of a hydrogen atom or a C₁₋₄alkyl group;
G is selected from the group consisting of C₄₋₇alkyl or a group of the formula (a), (b), (c) or (d) 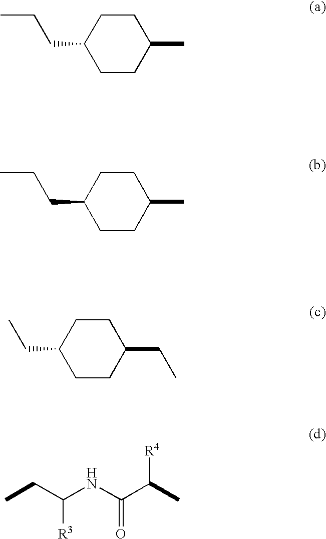
Wherein,
R³ and R⁴ are, independently, selected from a group consisting of a hydrogen, C₁₋₄alkyl, aryl, or C₁₋₂alkylaryl;
A is selected from a group consisting of an optionally substituted C₁₋₆alkyl
or a group of the formula (e), (f), (g) or (h): 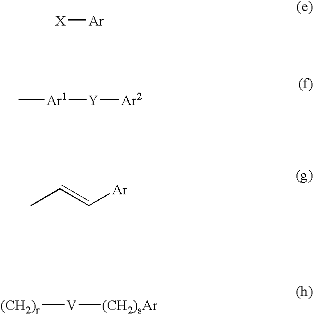
wherein
X is selected from a group consisting of a bond, NR², O or S;
Ar is selected from a group consisting of an optionally substituted phenyl ring or an optionally substituted 5- or 6-membered aromatic heterocyclic ring; or an optionally substituted bicyclic or heterobicyclic ring system;
Ar¹ and Ar² are, independently, selected from a group consisting of an optionally substituted phenyl ring or an optionally substituted 5- or 6-membered aromatic heterocyclic ring; and
Y is selected from a group consisting of a bond, —NHCO—, —CONH—, —CH₂—, or —(CH₂)ₘY¹ (CH₂)ₙ—, wherein Y¹ is selected from a group consisting of O, S, SO₂, or CO and m and n each represent zero or 1 such that the sum of m+n is zero or 1; provided that when A represents a group of formula (a), any substituent present in Ar ortho to the carboxamide moiety is necessarily a hydrogen or a methoxy group;
r and s are, independently, selected from a group consisting of an integer from zero to 3 such that the sum of r and s is equal to an integer from 1 to 6;
V is selected from a group consisting of a bond, O, S, —NHCO—, —CONH—, CHNHCOR³;
and salts thereof.

2. A compound according to claim 1 consisting of the group selected from:
2,8-dimethyl-N-(trans-4-{2-[7-(2-methylpropanoyl)-1,2,4,5-tetrahydro-3H-3-benzazepin-3-yl]ethyl}cyclohexyl)-5-quinolinecarboxamide;
2,8-Dimethyl-quinoline-5-carboxylic acid{4-[2-(7-butyryl-1,2,4,5-tetrahydro-3H-3-benzazepin-3-yl)-ethyl]-cyclohexyl}-amide;
8-Methoxy-2-methyl-quinoline-5-carboxylic acid{4-[2-(7-butyryl-1,2,4,5-tetrahydro-3H-3-benzazepin3-yl)-ethyl]-cyclohexyl}-amide;
8-Chloro-2-methyl-quinoline-5-carboxylic acid{4-[2-(7-butyryl-1,2,4,5-tetrahydro-3H-3-benzazepin3-yl)-ethyl]-cyclohexyl}-amide;
N-(trans-4-{2-[7-(2-methylpropanoyl)-1,2,4,5-tetrahydro-3H-3-benzazepin-3-yl]ethyl}cyclohexyl)-5-quinoxalinecarboxamide;
N-(trans-4-{2-[7-(2-methylpropanoyl)-1,2,4,5-tetrahydro-3H-3-benzazepin-3-yl]ethyl}cyclohexyl)-2,2-diphenylacetamide;
2-Methyl-quinoline-5-carboxylic acid{4-[2-(7-butyryl-1,2,4,5-tetrahydro-3H-3-benzazepin3-yl)-ethyl]-cyclohexyl}-amide;
N-(trans-4-{2-[7-(2-methylpropanoyl)-1,2,4,5-tetrahydro-3H-3-benzazepin-3-yl]ethyl}cyclohexyl)-1H-indole-2-carboxamide;
1,1-dimethylethyl 2-{[(trans-4-{2-[7-(2-methylpropanoyl)-1,2,4,5-tetrahydro-3H-3-benzazepin-3-yl]ethyl}cyclohexyl)amino]carbonyl}benzoate;
8-chloro-2-methyl-N-(trans-4-{2-[7-(2-methylpropanoyl)-1,2,4,5-tetrahydro-3H-3-benzazepin-3-yl]ethyl}cyclohexyl)-5-quinolinecarboxamide;
2-methyl-8-(methyloxy)-N-(trans-4-{2-[7-(2-methylpropanoyl)-1,2,4,5-tetrahydro-3H-3-benzazepin-3-yl]ethyl}cyclohexyl)-5-quinolinecarboxamide;
8-chloro-N-(trans-4-{2-[7-(2-methylpropanoyl)-1,2,4,5-tetrahydro-3H-3-benzazepin-3-yl]ethyl}cyclohexyl)-5-quinolinecarboxamide;

3. A compound according to claim 1 consisting of the group selected from:
1,1-dimethylethyl((1S)-2-[(trans-4-{2-[7-(2-methylpropanoyl)-1,2,4,5-tetrahydro-3H-3-benzazepin-3-yl]ethyl}cyclohexyl)amino]-2-oxo-1-{[(phenylmethyl)oxy]methyl}ethyl)carbamate formate;
N-(trans-4-{2-[7-(2-methylpropanoyl)-1,2,4,5-tetrahydro-3H-3-benzazepin-3-yl]ethyl}cyclohexyl)-N′-(phenylmethyl)butanediamide;
N-{5-[(trans-4-{2-[7-(2-methylpropanoyl)-1,2,4,5-tetrahydro-3H-3-benzazepin-3-yl]ethyl}cyclohexyl)amino]-5-oxopentyl}benzamide;
N-(trans-4-{2-[7-(2-methylpropanoyl)-1,2,4,5-tetrahydro-3H-3-benzazepin-3-yl]ethyl}cyclohexyl)-2-[(methylsulfonyl)amino]benzamide;
N¹-(trans-4-{2-[7-(2-methylpropanoyl)-1,2,4,5-tetrahydro-3H-3-benzazepin-3-yl]ethyl}cyclohexyl)-O-(phenylmethyl)-L-serinamide;
N-(trans-4-{2-[7-(2-methylpropanoyl)-1,2,4,5-tetrahydro-3H-3-benzazepin-3-yl]ethyl}cyclohexyl)-1-isoquinolinecarboxamide formate;
N-(trans-4-{2-[7-(2-methylpropanoyl)-1,2,4,5-tetrahydro-3H-3-benzazepin-3-yl]ethyl}cyclohexyl)-1,6-naphthyridine-2-carboxamide;
1,1-dimethylethyl({3-[(trans-4-{2-[7-(2-methylpropanoyl)-1,2,4,5-tetrahydro-3H-3-benzazepin-3-yl]ethyl}cyclohexyl)amino]-3-oxopropyl}thio)acetate;
2-(dimethylamino)-N-(trans-4-{2-[7-(2-methylpropanoyl)-1,2,4,5-tetrahydro-3H-3-benzazepin-3-yl]ethyl}cyclohexyl)benzamide;
N-(trans-4-{2-[7-(2-methylpropanoyl)-1,2,4,5-tetrahydro-3H-3-benzazepin-3-yl]ethyl}cyclohexyl)-3,3-diphenylpropanamide;
N-(trans-4-{2-[7-(2-methylpropanoyl)-1,2,4,5-tetrahydro-3H-3-benzazepin-3-yl]ethyl}cyclohexyl)-2-(2-naphthalenyl)acetamide formate;
(2E)-3-(1H-imidazol-4-yl)-N-(trans-4-{2-[7-(2-methylpropanoyl)-1,2,4,5-tetrahydro-3H-3-benzazepin-3-yl]ethyl}cyclohexyl)-2-propenamide;
N-(trans-4-{2-[7-(2-methylpropanoyl)-1,2,4,5-tetrahydro-3H-3-benzazepin-3-yl]ethyl}cyclohexyl)-6-(1H-pyrrol-1-yl)-3-pyridinecarboxamide;
4-amino-N-(trans-4-{2-[7-(2-methylpropanoyl)-1,2,4,5-tetrahydro-3H-3-benzazepin-3-yl]ethyl}cyclohexyl)butanamide;
N-(trans-4-{2-[7-(2-methylpropanoyl)-1,2,4,5-tetrahydro-3H-3-benzazepin-3-yl]ethyl}cyclohexyl)-4-pyridinecarboxamide formate;
1,1-dimethylethyl {4-[(trans-4-{2-[7-(2-methylpropanoyl)-1,2,4,5-tetrahydro-3H-3-benzazepin-3-yl]ethyl}cyclohexyl)amino]-4-oxobutyl}carbamate;
3-[3,4-bis(methyloxy)phenyl]-N-(trans-4-(2-[7-(2-methylpropanoyl)-1,2,4,5-tetrahydro-3H-3-benzazepin-3-yl]ethyl}cyclohexyl)propanamide;
N-{2-[(trans-4-{2-[7-(2-methylpropanoyl)-1,2,4,5-tetrahydro-3H-3-benzazepin-3-yl]ethyl}cyclohexyl)amino]-2-oxoethyl}-3-pyridinecarboxamide formate;
N-(trans-4-{2-[7-(2-methylpropanoyl)-1,2,4,5-tetrahydro-3H-3-benzazepin-3-yl]ethyl}cyclohexyl)-1-phenylcyclopentanecarboxamide;
2-[4-(dimethylamino)phenyl]-N-(trans-4-{2-[7-(2-methylpropanoyl)-1,2,4,5-tetrahydro-3H-3-benzazepin-3-yl]ethyl}cyclohexyl)acetamide;
N-(trans-4-{2-[7-(2-methylpropanoyl)-1,2,4,5-tetrahydro-3H-3-benzazepin-3-yl]ethyl}cyclohexyl)-1,8-naphthyridine-2-carboxamide;
N-(trans-4-{2-[7-(2-methylpropanoyl)-1,2,4,5-tetrahydro-3H-3-benzazepin-3-yl]ethyl}cyclohexyl)-2-(4-pyridinyl)acetamide;
N-(trans-4-{2-[7-(2-methylpropanoyl)-1,2,4,5-tetrahydro-3H-3-benzazepin-3-yl]ethyl}cyclohexyl)-4-oxo-4-phenyl-2-butenamide;
2-(5-hydroxy-1H-benzimidazol-1-yl)-N-(trans-4-{2-[7-(2-methylpropanoyl)-1,2,4,5-tetrahydro-3H-3-benzazepin-3-yl]ethyl}cyclohexyl)acetamide;
N-(trans-4-{2-[7-(2-methylpropanoyl)-1,2,4,5-tetrahydro-3H-3-benzazepin-3-yl]ethyl}cyclohexyl)-2-pyrazinecarboxamide;
1-methyl-N-(trans-4-{2-[7-(2-methylpropanoyl)-1,2,4,5-tetrahydro-3H-3-benzazepin-3-yl]ethyl}cyclohexyl)-1H-indole-2-carboxamide;
N-(trans-4-{2-[7-(2-methylpropanoyl)-1,2,4,5-tetrahydro-3H-3-benzazepin-3-yl]ethyl}cyclohexyl)-2-(3-pyridinyl)-1,3-thiazole-4-carboxamide;
1,1-dimethylethyl {(1R)-2-[(trans-4-{2-[7-(2-methylpropanoyl)-1,2,4,5-tetrahydro-3H-3-benzazepin-3-yl]ethyl}cyclohexyl)amino]-2-oxo-1-phenylethyl}carbamate;
N-(trans-4-(2-[7-(2-methylpropanoyl)-1,2,4,5-tetrahydro-3H-3-benzazepin-3-yl]ethyl}cyclohexyl)-3-pyridinecarboxamide 1-oxide formate;
3-(dimethylamino)-N-(trans-4-{2-[7-(2-methylpropanoyl)-1,2,4,5-tetrahydro-3H-3-benzazepin-3-yl]ethyl}cyclohexyl)benzamide;
N-(trans-4-(2-[7-(2-methylpropanoyl)-1,2,4,5-tetrahydro-3H-3-benzazepin-3-yl]ethyl}cyclohexyl)-1H-indole-2-carboxamide;
3-(1H-indol-3-yl)-N-(trans-4-{2-[7-(2-methylpropanoyl)-1,2,4,5-tetrahydro-3H-3-benzazepin-3-yl]ethyl}cyclohexyl)propanamide;
N-(trans-4-{2-[7-(2-methylpropanoyl)-1,2,4,5-tetrahydro-3H-3-benzazepin-3-yl]ethyl}cyclohexyl)-2-quinolinecarboxamide;
N-(trans-4-(2-[7-(2-methylpropanoyl)-1,2,4,5-tetrahydro-3H-3-benzazepin-3-yl]ethyl}cyclohexyl)-2-(1H-pyrrol-1-yl)benzamide;
1,1-dimethylethyl [(1R)-2-[(trans-4-{2-[7-(2-methylpropanoyl)-1,2,4,5-tetrahydro-3H-3-benzazepin-3-yl]ethyl}cyclohexyl)amino]-2-oxo-1-(3-pyridinylmethyl)ethyl]carbamate formate;
N-(trans-4-{2-[7-(2-methylpropanoyl)-1,2,4,5-tetrahydro-3H-3-benzazepin-3-yl]ethyl}cyclohexyl)-1H-indole-3-carboxamide formate;
5-methyl-N-(trans-4-{(2-[7-(2-methylpropanoyl)-1,2,4,5-tetrahydro-3H-3-benzazepin-3-yl]ethyl}cyclohexyl)-2-phenyl-2H-1,2,3-triazole-4-carboxamide;
N³[(4-methylphenyl)sulfonyl]-N-(trans-4-{2-[7-(2-methylpropanoyl)-1,2,4,5-tetrahydro-3H-3-benzazepin-3-yl]ethyl}cyclohexyl)-beta-alaninamide formate;
(2R)-2-amino-N-(trans-4-{2-[7-(2-methylpropanoyl)-1,2,4,5-tetrahydro-3H-3-benzazepin-3-yl]ethyl}cyclohexyl)-2-phenylethanamide;
N-(trans-4-{2-[7-(2-methylpropanoyl)-1,2,4,5-tetrahydro-3H-3-benzazepin-3-yl]ethyl}cyclohexyl)-2-(2-pyrimidinylthio)acetamide formate;
N-(trans-4-{2-[7-(2-methylpropanoyl)-1,2,4,5-tetrahydro-3H-3-benzazepin-3-yl]ethyl}cyclohexyl)-4-(1H-pyrrol-1-yl)benzamide;
N¹-(trans-4-{2-[7-(2-methylpropanoyl)-1,2,4,5-tetrahydro-3H-3-benzazepin-3-yl]ethyl}cyclohexyl)-3-(3-pyridinyl)-D-alaninamide;
(3E)-N-(trans-4-{2-[7-(2-methylpropanoyl)-1,2,4,5-tetrahydro-3H-3-benzazepin-3-yl]ethyl}cyclohexyl)-4-phenyl-3-butenamide;
2-(1H-indol-3-yl)-N-(trans-4-{2-[7-(2-methylpropanoyl)-1,2,4,5-tetrahydro-3H-3-benzazepin-3-yl]ethyl}cyclohexyl)acetamide;
N-(trans-4-{2-[7-(2-methylpropanoyl)-1,2,4,5-tetrahydro-3H-3-benzazepin-3-yl]ethyl}cyclohexyl)-2-[(phenylmethyl)thio]acetamide;
4-[4-(methyloxy)phenyl]-N-(trans-4-{2-[7-(2-methylpropanoyl)-1,2,4,5-tetrahydro-3H-3-benzazepin-3-yl]ethyl}cyclohexyl)butanamide;
2-Methyl-quinoline-5-carboxylic acid{4-[7-(2-methyl-propanoyl)-1,2,4,5-tetrahydro-3H-3-benzazepin-3-yl]-butyl}-amide;
2-Methyl-quinoline-5-carboxylic acid{8-[7-(2-methyl-propanoyl)-1,2,4,5-tetrahydro-3H-3-benzazepin-3-yl]-octyl}-amide;
2-Methyl-quinoline-5-carboxylic acid [({4-[7-(2-methyl-propanoyl)-1,2,4,5-tetrahydro-3H-3-benzazepin-3-ylmethyl]-cyclohexylmethyl}-carbamoyl)-methyl]-amide;
(R)-1-[1-(2-Methyl-quinolin-5-yl)-methanoyl)-pyrrolidine-2-carboxylic acid{4-[7-(2-methyl-propanoyl)-1,2,4,5-tetrahydro-3H-3-benzazepin-3-ylmethyl]-cyclohexylmethyl}-amide2-Methyl-quinoline-5-carboxylic acid;
[2-({4-[7-(2-methyl-propanoyl)-1,2,4,5-tetrahydro-3H-3-benzazepin-3-ylmethyl]-cyclohexylmethyl}-carbamoyl)-ethyl]-amide;
2,8-Dimethyl-quinoline-5-carboxylic acid{4-[2-(7-butyryl-1,2,4,5-tetrahydro-3H-3-benzazepin-3-yl)-ethyl]-cyclohexyl}-amide;
8-Methoxy-2-methyl-quinoline-5-carboxylic acid{4-[2-(7-butyryl-1,2,4,5-tetrahydro-3H-3-benzazepin3-yl)-ethyl]-cyclohexyl}-amide;
8-Chloro-2-methyl-quinoline-5-carboxylic acid{4-[2-(7-butyryl-1,2,4,5-tetrahydro-3H-3-benzazepin3-yl)-ethyl]-cyclohexyl}-amide;
2-Methyl-quinoline-5-carboxylic acid{4-[2-(7-butyryl-1,2,4,5-tetrahydro-3H-3-benzazepin3-yl)-ethyl]-cyclohexyl}-amide;
N-{4-[2-(7-Acetyl-1,2,4,5-tetrahydro-3H-3-benzazepin-3-yl)-ethyl]-cyclohexyl}-4-fluoro-benzamide;
N-{4-[2-(7-Acetyl-1,2,4,5-tetrahydro-3H-3-benzazepin-3-yl)-ethyl]-cyclohexyl}-3-(5-methyl-[1,2,4]oxadiazol-3-yl)-benzamide;
2-Methyl-quinoline-5-carboxylic acid{4-[2-(7-(2-methyl-propanoyl)-1,2,4,5-tetrahydro-3H-3-benzazepin3-yl)-ethyl]-cyclohexyl}-amide;
1H-pyrrolo[2,3-b]pyridine-3-carboxylic acid-{4-[2-(7-Acetyl-1,2,4,5-tetrahydro-3H-3-benzazepin-3-yl)-ethyl]-cyclohexyl}-amide;
(E)-N-{4-[2-(7-Acetyl-1,2,4,5-tetrahydro-3H-3-benzazepin-3-yl)-ethyl]-cyclohexyl}-3-(4-fluorophenyl)-acrylamide;
(E)-N-{4-[2-(7-Acetyl-1,2,4,5-tetrahydro-3H-3-benzazepin-3-yl)-ethyl]-cyclohexyl}-3-(3-methoxyphenyl)-acrylamide;
(E)-N-{4-[2-(7-Acetyl-1,2,4,5-tetrahydro-3H-3-benzazepin-3-yl)-ethyl]-cyclohexyl}-3-(2-cyanophenyl)-acrylamide;
(E)-N-{4-[2-(7-Acetyl-1,2,4,5-tetrahydro-3H-3-benzazepin-3-yl)-ethyl]-cyclohexyl}-3-(2-acetylphenyl)-acrylamide;
(E)-N-{4-[2-(7-Acetyl-1,2,4,5-tetrahydro-3H-3-benzazepin-3-yl)-ethyl]-cyclohexyl}-3-(3-thiophenyl)-acrylamide;
(E)-N-{4-[2-(7-Acetyl-1,2,4,5-tetrahydro-3H-3-benzazepin-3-yl)-ethyl]-cyclohexyl}-3-(8-(1,2-dihydro-2-oxo)-quinolinyl)-acrylamide;
(E)-N-{4-[2-(7-Acetyl-1,2,4,5-tetrahydro-3H-3-benzazepin-3-yl)-ethyl]-cyclohexyl}-3-(3-benzothiophenyl)-acrylamide;
(E)-N-{4-[2-(7-Acetyl-1,2,4,5-tetrahydro-3H-3-benzazepin-3-yl)-ethyl]-cyclohexyl}-3-(3-benzothiophenyl)-acrylamide;
(E)-3-(4-Acetylamino-phenyl)-N-{4-[2-(7-acetyl-1,2,4,5-tetrahydro-3H-3-benzazepin-3-yl)-ethyl]-cyclohexyl}-acrylamide;
N-{4-[2-(7-Acetyl-1,2,4,5-tetrahydro-3H-3-benzazepin-3-yl)-ethyl]-cyclohexyl}-2-(2-aminobenzothiazol-6-yl)-acetamide;
4-Oxo-1,4-dihydro-quinoline-8-carboxylic acid-{4-[2-(7-Acetyl-1,2,4,5-tetrahydro-3H-3-benzazepin-3-yl)-ethyl]-cyclohexyl}-amide;
N-{4-[2-(7-Acetyl-1,2,4,5-tetrahydro-3H-3-benzazepin-3-yl)-ethyl]-cyclohexyl}-2-(benzo[b]thiophen-2-yl)-acetamide
and pharmaceutically acceptable salts thereof.

4. A pharmaceutical composition for the treatment of muscarinic acetylcholine receptor mediated diseases comprising a compound according to claim 1 and a pharmaceutically acceptable carrier thereof.

5. A method of inhibiting the binding of acetylcholine to its receptors in a mammal in need thereof comprising administering a safe and effective amount of a compound according to claim 1.

6. A method of treating a muscarinic acetylcholine receptor mediated disease, wherein acetylcholine binds to said receptor, comprising administering a safe and effective amount of a compound according to claim 1.

7. A method according to claim 6 wherein the disease is selected from the group consisting of chronic obstructive lung disease, chronic bronchitis, asthma, chronic respiratory obstruction, pulmonary fibrosis, pulmonary emphysema and allergic rhinitis.

8. A method according to claim 7 wherein administration is via inhalation via the mouth or nose.

9. A method according to claim 8 wherein administration is via a medicament dispenser selected from a reservoir dry powder inhaler, a multi-dose dry powder inhaler or a metered dose inhaler.

10. A method according to claim 9 wherein the compound is administered to a human and has a duration of action of 12 hours or more for a 1 mg dose.

11. A method according to claim 10 wherein the compound has a duration of action of 24 hours or more.

12. A method according to claim 11 wherein the compound has a duration of action of 36 hours or more.
